SEQUENCE LISTING (36 sequences)

SEQ ID NO: 1 - DNA, 5108 bp, Homo sapiens
gccgtactgccttttttcccctctttcattctttctctccgtctttttctcccccctctgcgcacgaaggatgtgcttctaggtggtgatctgccctcctctctctcttttatcatttctcccccgccgccggcgagttgactctttccctatgtgtgtgaggcggcggcggcagcagcagcagcagcggctccggcggcggcagcagcggcagcagcgacttcagcggcggcggcggcgctagacgcagcggctccgggcccgacccggcggcttcggcggcggctccggcggcagcggcggcccgggcggcccgcagggaacggcgagcggcctccacccagcgactgcgggcggcggcggccggagagagcgaggcgcgcgccggacgcccggggcaggcggcggcggcggcggcccagcgccaggacgacgccgcgcagcgcccgacgcggaccactttcatgctgattcccccggacccgggcagcgctccggccactccgcgggccgccggcctccgccccggcctgcctggctccctgggcgcgcccgcacccggcgcctccgatctcctagtcctcctgatttcgatggctttcctgaatggctgactgtgggctgccctggacttggcccccggacagtcgcctctcctcctcctctacctcctccttcaccaccacctcctcttcctcctcctcctcctcctcctcctccgccaactcctcggctgcacaccagctctaagagcgagagtgaacgagagagggagggagagagtgagagcgagcgagatctttggagagatttttttttttgcctcctacttctgtcttgaagccagacaatcgacttcagctctccctcccctccctctttctccacgttctgctcccactcgctctcctgtccccttcccctcccctcccggcggaaagccccccgaaaccaacaaagctgagccgagagaaacaaacaaaacaaacacaccgggccagacaagccatcgacaaaactttgcaaaagcaaaaacaaaaaaggaaaaactaaccaacctcaaccaaccagcccccgagccacccggggcgccctcccgcgccctcttgcaccctcgcacacacaaaaggcggcgcgccggagcccgagacccggggagccgccgccgccccgccgccgcccgcagccaggggagcaggaagtccggacgcagcccccatagatatggcaatggtagtcagcacgtggcgcgacccccaggacgaggtgcccggctcacagggcagccaggcctcgcaggcgccgcccgtgcccggcccgccgcccggcgccccgcacacgccacagacgcccggccaagggggcccagccagcacgccagcccagacggcggccggtggccagggcggccctggcggcccgggtagcgacaagcagcagcagcagcaacacatcgagtgcgtggtgtgcggagacaagtcgagcggcaagcactacggccagttcacgtgcgagggctgcaagagcttcttcaagcgcagcgtgcggaggaacctgagctacacgtgccgcgccaaccggaactgtcccatcgaccagcaccatcgcaaccagtgccagtactgccgcctcaaaaagtgcctcaaagtgggcatgagacgggaagcggtgcagaggggcaggatgccgccgacccagccgacccacgggcagttcgcgctgaccaacggggatcccctcaactgccactcgtacctgtccggatatatttccctgctgttgcgcgcggagccctatcccacgtcgcgcttcggcagccaatgcatgcagcccaacaacatcatgggtatcgagaacatttgcgaactggccgcgaggatgctcttcagcgccgtcgagtgggcccggaacatccccttcttccccgacctgcagatcacggaccaggtggccctgcttcgcctcacctggagcgagctgtttgtgttgaatgcggcgcagtgctccatgcccctccacgtcgccccgctcctggccgccgccggcctgcatgcttcgcccatgtccgccgaccgggtggtcgcctttatggaccacatacggatcttccaagagcaagtggagaagctcaaggcgctgcacgttgactcagccgagtacagctgcctcaaggccatagtcctgttcacctcagatgcctgtggtctctctgatgtagcccatgtggaaagcttgcaggaaaagtctcagtgtgctttggaagaatacgttaggagccagtaccccaaccagccgacgagattcggaaagcttttgcttcgcctcccttccctccgcaccgtctcctcctcagtcatagagcaattgtttttcgtccgtttggtaggtaaaacccccatcgaaaccctcatccgggatatgttactgtccggcagcagttttaactggccgtatatggcaattcaataaataaataaaataagaagggggagtgaaacagagaaagaaaaggcaaaagactggtttgtttgcttaatttccttctgttaagaaaggatataaaaggatgttacaagtttgctaaaagaagagaggggaagaatttaatggactgtgaatttcaaaaaaaaaaaaaaagactgtcaaatgaacttttacagaatgcattaaaaaaaaaaaaaaactcctgtgtcggtcagaacaacttgctacttatcatttttgtataaaaaggaaattagtctttttctttttttggtaaatttttgaaaaatattgctaaaagtgcatttaaggagattgggagacaattagcagaatggagaaagtaagtcttttttttttccaaattattaattgtcctgtgtctatgtacctctagctgttcttttttgtacttttctggttccaaaccagtttattctgtggttctataataagttttgatataatcttggcttcttaaaaactgtgtatcattaaaatatatgttctgcaagaattaaaactgagtccatgaaaataccataggaagacataaaactttaaaaggcaactcaaagatgatggaaacgcacttacaagtggtgaccaaaatttttaggtgaagtcgagcactctaattagagaactggaggaaccacatataacacttaacttcccctaccctgcccctccccaaaagaaaccatgacaaacctagcttttaaaaaatattttaagaaagagaatgaactgtggaatttattggcagccaaggaatgtgtccaagacacatgctgaggttttgaataaaaagtgaacttttgtaatttgaattgggtcccgcttagttcttgaattgttatgaaaatcctatatctgtttgtatatttgcaaaccctttgtattataattgttgatattttccctttttaaaaaataccattgaaatcagcatgacaaaaataacactgttggcacttataggtaacgtgattgattcagtatcttagagtttacagtttgtgttttaaaaaaactgaaggttttttttttaagtgcaacatttctgtatactgtaaaagttataataactgaactgtttggtcgagtctttgtgtgttatattccaaggaaaattgaaagtattcagaaattaaaatattatttgatatctgaaacctggctgtccccactcactgtctttacatctagaagagcccctgtgagctctcgcttagctggccgggcggggggtggtgggggggggcatttgtttactcccctcagtcagtttgttcaaaggtggactactgtatttgcctgtttaatttgggtgtgtgtgtgttggggggggagctgaagttaatggtttatctatggtttaggaagtgccatactgatatagtaaaccacccccattcacctaatcctccttttaattaaaaatggattttccaggaaaaaaaaaaaggcccttatatttgtcacacttaagtgcctgcttagggaaggtattgtgaaaaagtattagaaatcttgagatcagtatctattttatgatcagaaaaaaatactcttttgtacatttctgacagttactcagaagatcgttcaagcaagctaatcacagcattgtaactagaggacagttgtttgcagtgagtttttccttaagtaggtacgattttttaaaatattctgtgattctactctagcgtggttgttgagagagtttcaaattcagtgatacaggttctaagactgaaaggtctacttttaatgtattatgataacttgcagttggtttccctctcccctccccccctttaccttcagtctgtgagagcatgaccacagggtcaagggaatcttttccattggagttatgtacataaaaacacatcgacattttgacatttcagattgtgtgctacaatctgtactgctcttgggatcctttgtccttagaagccaaattaaggaagagaaagcaggacagagaaaaagaaagaaggaaggagggaaactttacagggtgtgctgatttggaagtagtaactatttcttttggagtctttttttcatttttcctctttctcttttcctggtttggaggaagctcggtgctgggagcttgcaattttgttcttattcaaggtttccaacccacccccccaccgccagtacttcatcatgttgtggtttaattctaattggtgggggggggggaggactagtgagggaggtgaaagaacagggataattttgtaaagtgtattaaacgttaatattcagatccagtcaatacatgcagaccagtaaaatctgatttgtgcagagttctccatctgactctcacttatttctgtagatatatacatatataaatacaagtatgttcttacggcacagtattgctgacctttagttcgaggttttgtcggttgttgttgattttcttcctcttgcaagtgctatccatgtgagtgtgtgaagtttctctaataagtaaaacacaggcccttttccttgtttgttttgtgttagtttattgtaaacagccatttgttgtaaattattattggcattaaattataatttatgattttcaaagcaaaagacaa

SEQ ID NO: 2 - DNA, 3869 bp, Homo sapiens
atgagagacaaggatcactccagacatctcctacctacggtttggggttttttttcttaaaggcgaggcttgcattcctcagcagctatgtacaaagctccctgaaaccttgtctctctaaagttagtgtgcagggttttccaaggctgagagagcctaatacatggggaagcacttccttgaggtggaagatctctcccttcacctttcctctttttccctgcaggctagtgcctactttttatcagtttgcacaatcgcttagataaacaccgaggaggagattctctttaattatcaaagacacatcttttcagggggccaacaaagcatttatttcacccgccaaactaaaggagagttattccagtttaggaggaagatgcaagcggtttgggaccttgaacaaggcaaatatggttttgcggtgcagaggggcaggatgccgccgacccagccgacccacgggcagttcgcgctgaccaacggggatcccctcaactgccactcgtacctgtccggatatatttccctgctgttgcgcgcggagccctatcccacgtcgcgcttcggcagccaatgcatgcagcccaacaacatcatgggtatcgagaacatttgcgaactggccgcgaggatgctcttcagcgccgtcgagtgggcccggaacatccccttcttccccgacctgcagatcacggaccaggtggccctgcttcgcctcacctggagcgagctgtttgtgttgaatgcggcgcagtgctccatgcccctccacgtcgccccgctcctggccgccgccggcctgcatgcttcgcccatgtccgccgaccgggtggtcgcctttatggaccacatacggatcttccaagagcaagtggagaagctcaaggcgctgcacgttgactcagccgagtacagctgcctcaaggccatagtcctgttcacctcagatgcctgtggtctctctgatgtagcccatgtggaaagcttgcaggaaaagtctcagtgtgctttggaagaatacgttaggagccagtaccccaaccagccgacgagattcggaaagcttttgcttcgcctcccttccctccgcaccgtctcctcctcagtcatagagcaattgtttttcgtccgtttggtaggtaaaacccccatcgaaaccctcatccgggatatgttactgtccggcagcagttttaactggccgtatatggcaattcaataaataaataaaataagaagggggagtgaaacagagaaagaaaaggcaaaagactggtttgtttgcttaatttccttctgttaagaaaggatataaaaggatgttacaagtttgctaaaagaagagaggggaagaatttaatggactgtgaatttcaaaaaaaaaaaaaaagactgtcaaatgaacttttacagaatgcattaaaaaaaaaaaaaaactcctgtgtcggtcagaacaacttgctacttatcatttttgtataaaaaggaaattagtctttttctttttttggtaaatttttgaaaaatattgctaaaagtgcatttaaggagattgggagacaattagcagaatggagaaagtaagtcttttttttttccaaattattaattgtcctgtgtctatgtacctctagctgttcttttttgtacttttctggttccaaaccagtttattctgtggttctataataagttttgatataatcttggcttcttaaaaactgtgtatcattaaaatatatgttctgcaagaattaaaactgagtccatgaaaataccataggaagacataaaactttaaaaggcaactcaaagatgatggaaacgcacttacaagtggtgaccaaaatttttaggtgaagtcgagcactctaattagagaactggaggaaccacatataacacttaacttcccctaccctgcccctccccaaaagaaaccatgacaaacctagcttttaaaaaatattttaagaaagagaatgaactgtggaatttattggcagccaaggaatgtgtccaagacacatgctgaggttttgaataaaaagtgaacttttgtaatttgaattgggtcccgcttagttcttgaattgttatgaaaatcctatatctgtttgtatatttgcaaaccctttgtattataattgttgatattttccctttttaaaaaataccattgaaatcagcatgacaaaaataacactgttggcacttataggtaacgtgattgattcagtatcttagagtttacagtttgtgttttaaaaaaactgaaggttttttttttaagtgcaacatttctgtatactgtaaaagttataataactgaactgtttggtcgagtctttgtgtgttatattccaaggaaaattgaaagtattcagaaattaaaatattatttgatatctgaaacctggctgtccccactcactgtctttacatctagaagagcccctgtgagctctcgcttagctggccgggcggggggtggtgggggggggcatttgtttactcccctcagtcagtttgttcaaaggtggactactgtatttgcctgtttaatttgggtgtgtgtgtgttggggggggagctgaagttaatggtttatctatggtttaggaagtgccatactgatatagtaaaccacccccattcacctaatcctccttttaattaaaaatggattttccaggaaaaaaaaaaaggcccttatatttgtcacacttaagtgcctgcttagggaaggtattgtgaaaaagtattagaaatcttgagatcagtatctattttatgatcagaaaaaaatactcttttgtacatttctgacagttactcagaagatcgttcaagcaagctaatcacagcattgtaactagaggacagttgtttgcagtgagtttttccttaagtaggtacgattttttaaaatattctgtgattctactctagcgtggttgttgagagagtttcaaattcagtgatacaggttctaagactgaaaggtctacttttaatgtatatatgataacttgcagttggtttccctctcccctccccccctttaccttcagtctgtgagagcatgaccacagggtcaagggaatcttttccattggagttatgtacataaaaacacatcgacattttgacatttcagattgtgtggctacaatctgtactgctcttgggatcctttgtccttagaagccaaattaaggaagagaaagcaggacagagaaaaagaaagaaggaaggagggaaactttacagggtgtgctgatttggaagtagtaactatttcttttggagtctttttttcatttttcctctttctcttttcctggtttggaggaagctcggtgctgggagcttgcaattttgttcttattcaaggtttccaacccacccccccaccgccagtacttcatcatgttgtggtttaattctaattggtgggggggggggaggactagtgagggaggtgaaagaacagggataattttgtaaagtgtattaaacgttaatattcagatccagtcaatacatgcagaccagtaaaatctgatttgtgcagagttctccatctgactctcacttatttctgtagatatatacatatataaatacaagtatgttcttacggcacagtattgctgacctttagttcgaggttttgtcggttgttgttgattttcttcctcttgcaagtgctatccatgtgagtgtgtgaagtttctctaataagtaaaacacaggcccttttccttgtttgttttgtgttagtttattgtaaacagccatttgttgtaaattattattggcattaaattataatttatgattttcaaagcaaaagacaa

SEQ ID NO: 3 - DNA, 3501 bp, Homo sapiens
ctccttccctcgtcctgggtcccggggtcctgggtacgtttggctagcctgctctggcggtgcagaggggcaggatgccgccgacccagccgacccacgggcagttcgcgctgaccaacggggatcccctcaactgccactcgtacctgtccggatatatttccctgctgttgcgcgcggagccctatcccacgtcgcgcttcggcagccaatgcatgcagcccaacaacatcatgggtatcgagaacatttgcgaactggccgcgaggatgctcttcagcgccgtcgagtgggcccggaacatccccttcttccccgacctgcagatcacggaccaggtggccctgcttcgcctcacctggagcgagctgtttgtgttgaatgcggcgcagtgctccatgcccctccacgtcgccccgctcctggccgccgccggcctgcatgcttcgcccatgtccgccgaccgggtggtcgcctttatggaccacatacggatcttccaagagcaagtggagaagctcaaggcgctgcacgttgactcagccgagtacagctgcctcaaggccatagtcctgttcacctcagatgcctgtggtctctctgatgtagcccatgtggaaagcttgcaggaaaagtctcagtgtgctttggaagaatacgttaggagccagtaccccaaccagccgacgagattcggaaagcttttgcttcgcctcccttccctccgcaccgtctcctcctcagtcatagagcaattgtttttcgtccgtttggtaggtaaaacccccatcgaaaccctcatccgggatatgttactgtccggcagcagttttaactggccgtatatggcaattcaataaataaataaaataagaagggggagtgaaacagagaaagaaaaggcaaaagactggtttgtttgcttaatttccttctgttaagaaaggatataaaaggatgttacaagtttgctaaaagaagagaggggaagaatttaatggactgtgaatttcaaaaaaaaaaaaaaagactgtcaaatgaacttttacagaatgcattaaaaaaaaaaaaaaactcctgtgtcggtcagaacaacttgctacttatcatttttgtataaaaaggaaattagtctttttctttttttggtaaatttttgaaaaatattgctaaaagtgcatttaaggagattgggagacaattagcagaatggagaaagtaagtcttttttttttccaaattattaattgtcctgtgtctatgtacctctagctgttcttttttgtacttttctggttccaaaccagtttattctgtggttctataataagttttgatataatcttggcttcttaaaaactgtgtatcattaaaatatatgttctgcaagaattaaaactgagtccatgaaaataccataggaagacataaaactttaaaaggcaactcaaagatgatggaaacgcacttacaagtggtgaccaaaatttttaggtgaagtcgagcactctaattagagaactggaggaaccacatataacacttaacttcccctaccctgcccctccccaaaagaaaccatgacaaacctagcttttaaaaaatattttaagaaagagaatgaactgtggaatttattggcagccaaggaatgtgtccaagacacatgctgaggttttgaataaaaagtgaacttttgtaatttgaattgggtcccgcttagttcttgaattgttatgaaaatcctatatctgtttgtatatttgcaaaccctttgtattataattgttgatattttccctttttaaaaaataccattgaaatcagcatgacaaaaataacactgttggcacttataggtaacgtgattgattcagtatcttagagtttacagtttgtgttttaaaaaaactgaaggttttttttttaagtgcaacatttctgtatactgtaaaagttataataactgaactgtttggtcgagtctttgtgtgttatattccaaggaaaattgaaagtattcagaaattaaaatattatttgatatctgaaacctggctgtccccactcactgtctttacatctagaagagcccctgtgagctctcgcttagctggccgggcggggggtggtgggggggggcatttgtttactcccctcagtcagtttgttcaaaggtggactactgtatttgcctgtttaatttgggtgtgtgtgtgttggggggggagctgaagttaatggtttatctatggtttaggaagtgccatactgatatagtaaaccacccccattcacctaatcctccttttaattaaaaatggattttccaggaaaaaaaaaaaggcccttatatttgtcacacttaagtgcctgcttagggaaggtattgtgaaaaagtattagaaatcttgagatcagtatctattttatgatcagaaaaaaatactcttttgtacatttctgacagttactcagaagatcgttcaagcaagctaatcacagcattgtaactagaggacagttgtttgcagtgagtttttccttaagtaggtacgattttttaaaatattctgtgattctactctagcgtggttgttgagagagtttcaaattcagtgatacaggttctaagactgaaaggtctacttttaatgtatatatgataacttgcagttggtttccctctcccctccccccctttaccttcagtctgtgagagcatgaccacagggtcaagggaatcttttccattggagttatgtacataaaaacacatcgacattttgacatttcagattgtgtggctacaatctgtactgctcttgggatcctttgtccttagaagccaaattaaggaagagaaagcaggacagagaaaaagaaagaaggaaggagggaaactttacagggtgtgctgatttggaagtagtaactatttcttttggagtctttttttcatttttcctctttctcttttcctggtttggaggaagctcggtgctgggagcttgcaattttgttcttattcaaggtttccaacccacccccccaccgccagtacttcatcatgttgtggtttaattctaattggtgggggggggggaggactagtgagggaggtgaaagaacagggataattttgtaaagtgtattaaacgttaatattcagatccagtcaatacatgcagaccagtaaaatctgatttgtgcagagttctccatctgactctcacttatttctgtagatatatacatatataaatacaagtatgttcttacggcacagtattgctgacctttagttcgaggttttgtcggttgttgttgattttcttcctcttgcaagtgctatccatgtgagtgtgtgaagtttctctaataagtaaaacacaggcccttttccttgtttgttttgtgttagtttattgtaaacagccatttgttgtaaattattattggcattaaattataatttatgattttcaaagcaaaagacaa

SEQ ID NO: 4 - DNA, 3962 bp, Homo sapiens
ggtccggagtcagataacagcctgggcccgagcctcgccggctttccccggcccttacaggccctgcccaggctccgctagtgccggccgcctgctccctgcctctcccggcttcctctctctttagccggcctctctctctccgccctctccctccgtctctttctccgagcacactgattagacagacgccagacctccgctctctgcttgtctctcactgggggggttccccgccgggctggggctggggcttcggggtttgtgggagagtcgttccggagtggccacaggccgtctggggtggaccctcgtgccttttgcaaaagcgcctcaccctccccccagactcgcccctcccgctccctctcctccaatcaataagaaatatcagctgtttagcagtaaagaagaaagatgccctcagaatgctacatcccgcccacagcgccggggaccccgaggcaaggtggccaattctgggtcctcggcggaccagccccgagcgggcctcggagcggtgcagaggggcaggatgccgccgacccagccgacccacgggcagttcgcgctgaccaacggggatcccctcaactgccactcgtacctgtccggatatatttccctgctgttgcgcgcggagccctatcccacgtcgcgcttcggcagccaatgcatgcagcccaacaacatcatgggtatcgagaacatttgcgaactggccgcgaggatgctcttcagcgccgtcgagtgggcccggaacatccccttcttccccgacctgcagatcacggaccaggtggccctgcttcgcctcacctggagcgagctgtttgtgttgaatgcggcgcagtgctccatgcccctccacgtcgccccgctcctggccgccgccggcctgcatgcttcgcccatgtccgccgaccgggtggtcgcctttatggaccacatacggatcttccaagagcaagtggagaagctcaaggcgctgcacgttgactcagccgagtacagctgcctcaaggccatagtcctgttcacctcagatgcctgtggtctctctgatgtagcccatgtggaaagcttgcaggaaaagtctcagtgtgctttggaagaatacgttaggagccagtaccccaaccagccgacgagattcggaaagcttttgcttcgcctcccttccctccgcaccgtctcctcctcagtcatagagcaattgtttttcgtccgtttggtaggtaaaacccccatcgaaaccctcatccgggatatgttactgtccggcagcagttttaactggccgtatatggcaattcaataaataaataaaataagaagggggagtgaaacagagaaagaaaaggcaaaagactggtttgtttgcttaatttccttctgttaagaaaggatataaaaggatgttacaagtttgctaaaagaagagaggggaagaatttaatggactgtgaatttcaaaaaaaaaaaaaaagactgtcaaatgaacttttacagaatgcattaaaaaaaaaaaaaaactcctgtgtcggtcagaacaacttgctacttatcatttttgtataaaaaggaaattagtctttttctttttttggtaaatttttgaaaaatattgctaaaagtgcatttaaggagattgggagacaattagcagaatggagaaagtaagtcttttttttttccaaattattaattgtcctgtgtctatgtacctctagctgttcttttttgtacttttctggttccaaaccagtttattctgtggttctataataagttttgatataatcttggcttcttaaaaactgtgtatcattaaaatatatgttctgcaagaattaaaactgagtccatgaaaataccataggaagacataaaactttaaaaggcaactcaaagatgatggaaacgcacttacaagtggtgaccaaaatttttaggtgaagtcgagcactctaattagagaactggaggaaccacatataacacttaacttcccctaccctgcccctccccaaaagaaaccatgacaaacctagcttttaaaaaatattttaagaaagagaatgaactgtggaatttattggcagccaaggaatgtgtccaagacacatgctgaggttttgaataaaaagtgaacttttgtaatttgaattgggtcccgcttagttcttgaattgttatgaaaatcctatatctgtttgtatatttgcaaaccctttgtattataattgttgatattttccctttttaaaaaataccattgaaatcagcatgacaaaaataacactgttggcacttataggtaacgtgattgattcagtatcttagagtttacagtttgtgttttaaaaaaactgaaggttttttttttaagtgcaacatttctgtatactgtaaaagttataataactgaactgtttggtcgagtctttgtgtgttatattccaaggaaaattgaaagtattcagaaattaaaatattatttgatatctgaaacctggctgtccccactcactgtctttacatctagaagagcccctgtgagctctcgcttagctggccgggcggggggtggtgggggggggcatttgtttactcccctcagtcagtttgttcaaaggtggactactgtatttgcctgtttaatttgggtgtgtgtgtgttggggggggagctgaagttaatggtttatctatggtttaggaagtgccatactgatatagtaaaccacccccattcacctaatcctccttttaattaaaaatggattttccaggaaaaaaaaaaaggcccttatatttgtcacacttaagtgcctgcttagggaaggtattgtgaaaaagtattagaaatcttgagatcagtatctattttatgatcagaaaaaaatactcttttgtacatttctgacagttactcagaagatcgttcaagcaagctaatcacagcattgtaactagaggacagttgtttgcagtgagtttttccttaagtaggtacgattttttaaaatattctgtgattctactctagcgtggttgttgagagagtttcaaattcagtgatacaggttctaagactgaaaggtctacttttaatgtatatatgataacttgcagttggtttccctctcccctccccccctttaccttcagtctgtgagagcatgaccacagggtcaagggaatcttttccattggagttatgtacataaaaacacatcgacattttgacatttcagattgtgtggctacaatctgtactgctcttgggatcctttgtccttagaagccaaattaaggaagagaaagcaggacagagaaaaagaaagaaggaaggagggaaactttacagggtgtgctgatttggaagtagtaactatttcttttggagtctttttttcatttttcctctttctcttttcctggtttggaggaagctcggtgctgggagcttgcaattttgttcttattcaaggtttccaacccacccccccaccgccagtacttcatcatgttgtggtttaattctaattggtgggggggggggaggactagtgagggaggtgaaagaacagggataattttgtaaagtgtattaaacgttaatattcagatccagtcaatacatgcagaccagtaaaatctgatttgtgcagagttctccatctgactctcacttatttctgtagatatatacatatataaatacaagtatgttcttacggcacagtattgctgacctttagttcgaggttttgtcggttgttgttgattttcttcctcttgcaagtgctatccatgtgagtgtgtgaagtttctctaataagtaaaacacaggcccttttccttgtttgttttgtgttagtttattgtaaacagccatttgttgtaaattattattggcattaaattataatttatgattttcaaagcaaaagacaa

SEQ ID NO: 5 - DNA, 4216 bp, Mus musculus
cgagggaaacaaacaaaacaaacacaccgggccagacaagcaatcgacaaaactttgcaaaagcaaaaacaaaaaaacaaaaaaggaaaaactaaccaacctcaaatcaactagccctgagccacccggggcgccctcccgcgccctctcgcaccctcgcacacacaaaaggcggcgcgccggagcccgagacccgggagccgccgccaccccgccgccgcccgcagccaggggagcagaagtccggacgcggcccccatagatatggcaatggtagtcagcacgtggcgcgacccccaggacgaggtgcccggctctcagggcagccaggcctcgcaggcgccgcccgtgccgggcccgccgcctggcgccccgcacacgccacagacgccgggccaagggggcccggccagcacgccggcccagacagcggctggcggccagggcggccctggcggcccgggcagcgacaagcagcagcagcagcagcacatcgagtgcgtggtgtgcggggacaagtcgagcggcaagcactacggccagttcacgtgcgagggctgcaagagcttcttcaagcgcagcgtgcggaggaacctgagctacacgtgccgcgccaaccggaactgtcccatcgaccagcaccaccgcaaccagtgccagtactgccgcctcaaaaagtgcctcaaagtgggcatgagacgggaagctgtacagagaggcaggatgcctcctacccagcctacccacgggcagtttgccctgaccaacggggaccccctcaactgccactcgtacctgtccggatatatttccctgctgctgcgcgcggagccctaccccacgtcgcgcttcggcagtcagtgcatgcagcctaacaacatcatgggcatcgagaacatttgcgaactggccgcacggatgctcttcagcgccgttgagtgggcccggaacatccccttcttccctgacctgcagatcacggaccaggtggccctccttcgcctcacctggagcgagctgttcgtgttgaatgcggcccagtgctccatgcccctccatgtcgccccgctccttgccgctgctggcctgcacgcttcacccatgtcagccgaccgggtggtcgcttttatggaccacatacggatcttccaagagcaagtggagaagctcaaggcactgcacgtcgactccgccgagtatagctgcctcaaggccatagtcctgttcacctcagatgcctgtggtctgtctgatgtagcccatgtggaaagcttgcaggaaaagtcccagtgtgctttggaagagtacgttaggagccagtaccccaaccagccaacacggttcggaaagctcttgcttcgtctcccttccctccgcacggtctcctcctcagtcatagagcaattgtttttcgtccgtttggtaggtaaaacccccatcgaaaccctcatccgggatatgttactgtccggcagcagttttaactggccatatatggcaattcaataaataaatcaatcaaaataagggggagtgaaacagagaaagaaaaggcaaaagactggttttgtttgcttaatttccttctgttaagaaaggatgttacaagtttgctaaaaagaagagaggggaagaatttaatggactgtgaatttcaaaaaggagagagagaaagagagagactgccaaatgaacttttacagaatgcattaaaaaaaaagaaagaaaacaactcctgtgttgggcagaacaacctgctacttatcatttttgtataaaaaggaaattagtctttttttctttttggtaaatttttgaaaaatattgctaaaagtgcatttaaggagattgggagaaaattagcagaatggacaaagtaagtcatttttttccaaattattaattgtcctgtgtctatgtacctctagttgttctttttttttttttttaacttttctggttccaaaccagtttattctgtggttcaataataagttttgatataatcttggcttcttaaaaactgtgtatcattaaaatatatgttctgcaagaattaaaactgagtccatgaaaatagcataggaaaacataaaactttaaaaggcaactcagagatggtggaaatgcacttacaagtggtggccaaattgtttttttttttttttttttaaggtaaagttgagcactctaattagcaagctgggggaatcacatcaacacttagcttccccacccccaccccataccatgacaaacctagctttttaaaaaaaatattttaagaaacagaaggaactgtggaatttattggcagccaaggaatgtgtccaagacacaagctgaggtttttgaataaaaagtgaacttttgtaatttgaattgggtcccccccccttagttcttgaattgttatgaatcctatatctgtttgtatatttgcaagccctttgtattataattgttgatatttcccctttttaaaaaataccattgaaatcagcatgacaaaataacactgttggcacttataggtaacgtgattgattcagtatcttagagtttacagtttgtgtttttaaaaaactgaaggttttttttttaagtgcaacatttctgtatactgtaaaagttataataactgaactgtttggtcgagtctttgtgtgttatattccaaggaaattgaaagtattcagaaattaaaatattatttgatatctgaaatctgcttggctgtccccactcactgtctttccacggagctgagcccctgtgagttctcgctgagccagcgggggccccatttgtttactccctcaatcagtttgttcaaaggtagactagtgtatttgcctgtttaatttgggtgtggtgtggggggggagctgaagttaatggtttagctatggtttaggaagtgccacactgatatagtaagccacccccattcacctaatcctacttttaattaaaaatggattttccaggaaaaaaataaggcccttatatttgtcacacttaagtgcctgcttagggaaggtattgtgaaaagtattagaaattttgagatcagtatctgttttatgatcagaaaaaaaatgctcttttgtacatttgtgacagttatgcagaggactgtccaagcaagctaatcacagaactgtaaatagagggcagttgtttgcaatgagtttttccttaagtaagtgtaatttttctttttctttttttcttttttttttaaaaatatcctgaggttctcatttagcgtggctgttgagaggattttgaatacagtgatgtagctgctagcgacgaagggtctgtttttcttgtatatacatgataacttgcagttgccctgcctttcccctccccctccctcttcagtctgttgagagcatggccacaggtcaagggaatctttaccattggagttatgtacataaaaaaaaaaaaccatgaacatttggacatttcagattatatagaaacaatctgtactgctctgggatcctttggtcttagaaaccatttttgggggggtggagagagagagagggagaggagagagagagagagagagagagagagagagagagagagagagagagagagagagagagagaataaagaaaactttacagggtgtgctgatttgggaagtcaactatttggttctgtcccttattctcttttcctggtttgggaagagctcactgctgggaacttgcaatttgttcttatttagactttccaagctgccctccctgacaatacttttaccatgttgtggtttaatcttaaaacgggggagggggctggtgacagaggtgaaagaaaggagatcagtttgccaagtgcattcaactttgatgctcagttctggttcatacatgcagacctgaaaactctgcctgatttaggcagagatctttatctgaccctcagcttccctctgtagatatatagatatataaatataaatatgaatataagtatgttttacagcacagcatctgacctgtagatggaggttttgttggttgtttattttcccctcttgcaagtgctacccatgtgagtgtgtgaagtttctctactaagtaaaacacaggcccttttccttgtttgctttgtgttagcttattgtaaacagccatttgttgtaaattattattggcattaaattataatttatgattttcaaagc

SEQ ID NO: 6 - DNA, 4393 bp, Mus musculus
ttatgcacttcgccgtattaacgctgccgcctgggcagagctcatgtgacccctccgtggattaacattctgctttaaaaaaatacctcttgttttctttttttttcctttcacttttgaaaccctgagagcactgtagggagtaggaaagtgtgggcaaggggcctttggccgctgctttccctctgcgccagttgggtctttgtgatataaaattatcccagagccgggaacagtgctctaccaatggcgcgctccgcgcctgggcgcgggctccgggttggagcgagccaatgccggggtttctttgtgtttctgcgagagcgactctcccggtcctgagtcagataacagcgggtgcctgagcctcgccggctttctccggtcgtcaccggccttgtctgggctccgcaagcgccccacgtctgctcccagcctctctcccgcttcctctcctctctgacggcctcactctttctctctccgcccttttctcccttgtctctcctcctccgagctgagctcagggatcaggcaaagacgccagaccaagactctgtctctcgccggggtttccttcctgggctggggttgaggttacaaggtttggggacagtcgttcggaggtggccacaggccatctggggtaaaccttaatgtcttgtgcaaaagcgtctcaccctccccctacattcccgtctcgttccttctccaatcaataagaaatatcagctatttagcagtttttaaaaagaaagaaatgaaatgaaacgaaaggtgccctaaggatatgctgcacctcgcttacagctccagggaccccattcaaagtgaccaattctgggtcctcggcggaccaagcctagatgggcctcacagctgtacagagaggcaggatgcctcctacccagcctacccacgggcagtttgccctgaccaacggggaccccctcaactgccactcgtacctgtccggatatatttccctgctgctgcgcgcggagccctaccccacgtcgcgcttcggcagtcagtgcatgcagcctaacaacatcatgggcatcgagaacatttgcgaactggccgcacggatgctcttcagcgccgttgagtgggcccggaacatccccttcttccctgacctgcagatcacggaccaggtggccctccttcgcctcacctggagcgagctgttcgtgttgaatgcggcccagtgctccatgcccctccatgtcgccccgctccttgccgctgctggcctgcacgcttcacccatgtcagccgaccgggtggtcgcttttatggaccacatacggatcttccaagagcaagtggagaagctcaaggcactgcacgtcgactccgccgagtatagctgcctcaaggccatagtcctgttcacctcagatgcctgtggtctgtctgatgtagcccatgtggaaagcttgcaggaaaagtcccagtgtgctttggaagagtacgttaggagccagtaccccaaccagccaacacggttcggaaagctcttgcttcgtctcccttccctccgcacggtctcctcctcagtcatagagcaattgtttttcgtccgtttggtaggtaaaacccccatcgaaaccctcatccgggatatgttactgtccggcagcagttttaactggccatatatggcaattcaataaataaatcaatcaaaataagggggagtgaaacagagaaagaaaaggcaaaagactggttttgtttgcttaatttccttctgttaagaaaggatgttacaagtttgctaaaaagaagagaggggaagaatttaatggactgtgaatttcaaaaaggagagagagaaagagagagactgccaaatgaacttttacagaatgcattaaaaaaaaagaaagaaaacaactcctgtgttgggcagaacaacctgctacttatcatttttgtataaaaaggaaattagtctttttttctttttggtaaatttttgaaaaatattgctaaaagtgcatttaaggagattgggagaaaattagcagaatggacaaagtaagtcatttttttccaaattattaattgtcctgtgtctatgtacctctagttgttctttttttttttttttaacttttctggttccaaaccagtttattctgtggttcaataataagttttgatataatcttggcttcttaaaaactgtgtatcattaaaatatatgttctgcaagaattaaaactgagtccatgaaaatagcataggaaaacataaaactttaaaaggcaactcagagatggtggaaatgcacttacaagtggtggccaaattgtttttttttttttttttttaaggtaaagttgagcactctaattagcaagctgggggaatcacatcaacacttagcttccccacccccaccccataccatgacaaacctagctttttaaaaaaaatattttaagaaacagaaggaactgtggaatttattggcagccaaggaatgtgtccaagacacaagctgaggtttttgaataaaaagtgaacttttgtaatttgaattgggtcccccccccttagttcttgaattgttatgaatcctatatctgtttgtatatttgcaagccctttgtattataattgttgatatttcccctttttaaaaaataccattgaaatcagcatgacaaaataacactgttggcacttataggtaacgtgattgattcagtatcttagagtttacagtttgtgtttttaaaaaactgaaggttttttttttaagtgcaacatttctgtatactgtaaaagttataataactgaactgtttggtcgagtctttgtgtgttatattccaaggaaattgaaagtattcagaaattaaaatattatttgatatctgaaatctgcttggctgtccccactcactgtctttccacggagctgagcccctgtgagttctcgctgagccagcgggggccccatttgtttactccctcaatcagtttgttcaaaggtagactagtgtatttgcctgtttaatttgggtgtggtgtggggggggagctgaagttaatggtttagctatggtttaggaagtgccacactgatatagtaagccacccccattcacctaatcctacttttaattaaaaatggattttccaggaaaaaaataaggcccttatatttgtcacacttaagtgcctgcttagggaaggtattgtgaaaagtattagaaattttgagatcagtatctgttttatgatcagaaaaaaaatgctcttttgtacatttgtgacagttatgcagaggactgtccaagcaagctaatcacagaactgtaaatagagggcagttgtttgcaatgagtttttccttaagtaagtgtaatttttctttttctttttttcttttttttttaaaaatatcctgaggttctcatttagcgtggctgttgagaggattttgaatacagtgatgtagctgctagcgacgaagggtctgtttttcttgtatatacatgataacttgcagttgccctgcctttcccctccccctccctcttcagtctgttgagagcatggccacaggtcaagggaatctttaccattggagttatgtacataaaaaaaaaaaaccatgaacatttggacatttcagattatatagaaacaatctgtactgctctgggatcctttggtcttagaaaccatttttgggggggtggagagagagagagggagaggagagagagagagagagagagagagagagagagagagagagagagagagagagagagagagaataaagaaaactttacagggtgtgctgatttgggaagtcaactatttggttctgtcccttattctcttttcctggtttgggaagagctcactgctgggaacttgcaatttgttcttatttagactttccaagctgccctccctgacaatacttttaccatgttgtggtttaatcttaaaacgggggagggggctggtgacagaggtgaaagaaaggagatcagtttgccaagtgcattcaactttgatgctcagttctggttcatacatgcagacctgaaaactctgcctgatttaggcagagatctttatctgaccctcagcttccctctgtagatatatagatatataaatataaatatgaatataagtatgttttacagcacagcatctgacctgtagatggaggttttgttggttgtttattttcccctcttgcaagtgctacccatgtgagtgtgtgaagtttctctactaagtaaaacacaggcccttttccttgtttgctttgtgttagcttattgtaaacagccatttgttgtaaattattattggcattaaattataatttatgattttcaaagcaaaa

SEQ ID NO: 7 - DNA, 4164 bp, Mus musculus
aaatggtggaatttggctgtgcctcggggttgtcctgctttgcaatattgcctatagttgttttcggttttctgctaagactgagccgggttgctccagcctccgactaaactcattaagttgggagatttttttttttttttcaattggaagggtgtttttaaagtctcctctttccagccccaaacaaggtgtaacaacgcactcttccttctaaggcatcagatgagagacaaggatcactccagacagctcctacctacggtttggggtttttttttcttaaaggcgaggcttgcattcctcagcagctatgtacaaagctccctgaagctgtctctctctctctaaagttagtgtgcaggcttttccaacggctgagagcgcctggtacacagggaagcagttccttgaggtggaagatctcttctttcacctttctttttccctgcagactaatgcctacttttttatcagtttgcacaatcgcttagataaacaccgaggaggagagtctctttaattatcaaagacacatcttttcagggggccaacaaagcatttatttcacccgccaaactaaaggagagttattccagtttagaaggaagatgcaagcggtttgggaccttgaacaaggcaaatatggttttgctgtacagagaggcaggatgcctcctacccagcctacccacgggcagtttgccctgaccaacggggaccccctcaactgccactcgtacctgtccggatatatttccctgctgctgcgcgcggagccctaccccacgtcgcgcttcggcagtcagtgcatgcagcctaacaacatcatgggcatcgagaacatttgcgaactggccgcacggatgctcttcagcgccgttgagtgggcccggaacatccccttcttccctgacctgcagatcacggaccaggtggccctccttcgcctcacctggagcgagctgttcgtgttgaatgcggcccagtgctccatgcccctccatgtcgccccgctccttgccgctgctggcctgcacgcttcacccatgtcagccgaccgggtggtcgcttttatggaccacatacggatcttccaagagcaagtggagaagctcaaggcactgcacgtcgactccgccgagtatagctgcctcaaggccatagtcctgttcacctcagatgcctgtggtctgtctgatgtagcccatgtggaaagcttgcaggaaaagtcccagtgtgctttggaagagtacgttaggagccagtaccccaaccagccaacacggttcggaaagctcttgcttcgtctcccttccctccgcacggtctcctcctcagtcatagagcaattgtttttcgtccgtttggtaggtaaaacccccatcgaaaccctcatccgggatatgttactgtccggcagcagttttaactggccatatatggcaattcaataaataaatcaatcaaaataagggggagtgaaacagagaaagaaaaggcaaaagactggttttgtttgcttaatttccttctgttaagaaaggatgttacaagtttgctaaaaagaagagaggggaagaatttaatggactgtgaatttcaaaaaggagagagagaaagagagagactgccaaatgaacttttacagaatgcattaaaaaaaaagaaagaaaacaactcctgtgttgggcagaacaacctgctacttatcatttttgtataaaaaggaaattagtctttttttctttttggtaaatttttgaaaaatattgctaaaagtgcatttaaggagattgggagaaaattagcagaatggacaaagtaagtcatttttttccaaattattaattgtcctgtgtctatgtacctctagttgttctttttttttttttttaacttttctggttccaaaccagtttattctgtggttcaataataagttttgatataatcttggcttcttaaaaactgtgtatcattaaaatatatgttctgcaagaattaaaactgagtccatgaaaatagcataggaaaacataaaactttaaaaggcaactcagagatggtggaaatgcacttacaagtggtggccaaattgtttttttttttttttttttaaggtaaagttgagcactctaattagcaagctgggggaatcacatcaacacttagcttccccacccccaccccataccatgacaaacctagctttttaaaaaaaatattttaagaaacagaaggaactgtggaatttattggcagccaaggaatgtgtccaagacacaagctgaggtttttgaataaaaagtgaacttttgtaatttgaattgggtcccccccccttagttcttgaattgttatgaatcctatatctgtttgtatatttgcaagccctttgtattataattgttgatatttcccctttttaaaaaataccattgaaatcagcatgacaaaataacactgttggcacttataggtaacgtgattgattcagtatcttagagtttacagtttgtgtttttaaaaaactgaaggttttttttttaagtgcaacatttctgtatactgtaaaagttataataactgaactgtttggtcgagtctttgtgtgttatattccaaggaaattgaaagtattcagaaattaaaatattatttgatatctgaaatctgcttggctgtccccactcactgtctttccacggagctgagcccctgtgagttctcgctgagccagcgggggccccatttgtttactccctcaatcagtttgttcaaaggtagactagtgtatttgcctgtttaatttgggtgtggtgtggggggggagctgaagttaatggtttagctatggtttaggaagtgccacactgatatagtaagccacccccattcacctaatcctacttttaattaaaaatggattttccaggaaaaaaataaggcccttatatttgtcacacttaagtgcctgcttagggaaggtattgtgaaaagtattagaaattttgagatcagtatctgttttatgatcagaaaaaaaatgctcttttgtacatttgtgacagttatgcagaggactgtccaagcaagctaatcacagaactgtaaatagagggcagttgtttgcaatgagtttttccttaagtaagtgtaatttttctttttctttttttcttttttttttaaaaatatcctgaggttctcatttagcgtggctgttgagaggattttgaatacagtgatgtagctgctagcgacgaagggtctgtttttcttgtatatacatgataacttgcagttgccctgcctttcccctccccctccctcttcagtctgttgagagcatggccacaggtcaagggaatctttaccattggagttatgtacataaaaaaaaaaaaccatgaacatttggacatttcagattatatagaaacaatctgtactgctctgggatcctttggtcttagaaaccatttttgggggggtggagagagagagagggagaggagagagagagagagagagagagagagagagagagagagagagagagagagagagagagagaataaagaaaactttacagggtgtgctgatttgggaagtcaactatttggttctgtcccttattctcttttcctggtttgggaagagctcactgctgggaacttgcaatttgttcttatttagactttccaagctgccctccctgacaatacttttaccatgttgtggtttaatcttaaaacgggggagggggctggtgacagaggtgaaagaaaggagatcagtttgccaagtgcattcaactttgatgctcagttctggttcatacatgcagacctgaaaactctgcctgatttaggcagagatctttatctgaccctcagcttccctctgtagatatatagatatataaatataaatatgaatataagtatgttttacagcacagcatctgacctgtagatggaggttttgttggttgtttattttcccctcttgcaagtgctacccatgtgagtgtgtgaagtttctctactaagtaaaacacaggcccttttccttgtttgctttgtgttagcttattgtaaacagccatttgttgtaaattattattggcattaaattataatttatgattttcaaagc

SEQ ID NO: 8 - DNA, 3622 bp, Mus musculus
aaaaagtgcctcaaagtgggcatgagacgggaaggtatcggcctctcatttcttcccccttcgcccgcggtcccggggctctgggtgcgtttggctagcctgctctggctgtacagagaggcaggatgcctcctacccagcctacccacgggcagtttgccctgaccaacggggaccccctcaactgccactcgtacctgtccggatatatttccctgctgctgcgcgcggagccctaccccacgtcgcgcttcggcagtcagtgcatgcagcctaacaacatcatgggcatcgagaacatttgcgaactggccgcacggatgctcttcagcgccgttgagtgggcccggaacatccccttcttccctgacctgcagatcacggaccaggtggccctccttcgcctcacctggagcgagctgttcgtgttgaatgcggcccagtgctccatgcccctccatgtcgccccgctccttgccgctgctggcctgcacgcttcacccatgtcagccgaccgggtggtcgcttttatggaccacatacggatcttccaagagcaagtggagaagctcaaggcactgcacgtcgactccgccgagtatagctgcctcaaggccatagtcctgttcacctcagatgcctgtggtctgtctgatgtagcccatgtggaaagcttgcaggaaaagtcccagtgtgctttggaagagtacgttaggagccagtaccccaaccagccaacacggttcggaaagctcttgcttcgtctcccttccctccgcacggtctcctcctcagtcatagagcaattgtttttcgtccgtttggtaggtaaaacccccatcgaaaccctcatccgggatatgttactgtccggcagcagttttaactggccatatatggcaattcaataaataaatcaatcaaaataagggggagtgaaacagagaaagaaaaggcaaaagactggttttgtttgcttaatttccttctgttaagaaaggatgttacaagtttgctaaaaagaagagaggggaagaatttaatggactgtgaatttcaaaaaggagagagagaaagagagagactgccaaatgaacttttacagaatgcattaaaaaaaaagaaagaaaacaactcctgtgttgggcagaacaacctgctacttatcatttttgtataaaaaggaaattagtctttttttctttttggtaaatttttgaaaaatattgctaaaagtgcatttaaggagattgggagaaaattagcagaatggacaaagtaagtcatttttttccaaattattaattgtcctgtgtctatgtacctctagttgttctttttttttttttttaacttttctggttccaaaccagtttattctgtggttcaataataagttttgatataatcttggcttcttaaaaactgtgtatcattaaaatatatgttctgcaagaattaaaactgagtccatgaaaatagcataggaaaacataaaactttaaaaggcaactcagagatggtggaaatgcacttacaagtggtggccaaattgtttttttttttttttttttaaggtaaagttgagcactctaattagcaagctgggggaatcacatcaacacttagcttccccacccccaccccataccatgacaaacctagctttttaaaaaaaatattttaagaaacagaaggaactgtggaatttattggcagccaaggaatgtgtccaagacacaagctgaggtttttgaataaaaagtgaacttttgtaatttgaattgggtcccccccccttagttcttgaattgttatgaatcctatatctgtttgtatatttgcaagccctttgtattataattgttgatatttcccctttttaaaaaataccattgaaatcagcatgacaaaataacactgttggcacttataggtaacgtgattgattcagtatcttagagtttacagtttgtgtttttaaaaaactgaaggttttttttttaagtgcaacatttctgtatactgtaaaagttataataactgaactgtttggtcgagtctttgtgtgttatattccaaggaaattgaaagtattcagaaattaaaatattatttgatatctgaaatctgcttggctgtccccactcactgtctttccacggagctgagcccctgtgagttctcgctgagccagcgggggccccatttgtttactccctcaatcagtttgttcaaaggtagactagtgtatttgcctgtttaatttgggtgtggtgtggggggggagctgaagttaatggtttagctatggtttaggaagtgccacactgatatagtaagccacccccattcacctaatcctacttttaattaaaaatggattttccaggaaaaaaataaggcccttatatttgtcacacttaagtgcctgcttagggaaggtattgtgaaaagtattagaaattttgagatcagtatctgttttatgatcagaaaaaaaatgctcttttgtacatttgtgacagttatgcagaggactgtccaagcaagctaatcacagaactgtaaatagagggcagttgtttgcaatgagtttttccttaagtaagtgtaatttttctttttctttttttcttttttttttaaaaatatcctgaggttctcatttagcgtggctgttgagaggattttgaatacagtgatgtagctgctagcgacgaagggtctgtttttcttgtatatacatgataacttgcagttgccctgcctttcccctccccctccctcttcagtctgttgagagcatggccacaggtcaagggaatctttaccattggagttatgtacataaaaaaaaaaaaccatgaacatttggacatttcagattatatagaaacaatctgtactgctctgggatcctttggtcttagaaaccatttttgggggggtggagagagagagagggagaggagagagagagagagagagagagagagagagagagagagagagagagagagagagagagagaataaagaaaactttacagggtgtgctgatttgggaagtcaactatttggttctgtcccttattctcttttcctggtttgggaagagctcactgctgggaacttgcaatttgttcttatttagactttccaagctgccctccctgacaatacttttaccatgttgtggtttaatcttaaaacgggggagggggctggtgacagaggtgaaagaaaggagatcagtttgccaagtgcattcaactttgatgctcagttctggttcatacatgcagacctgaaaactctgcctgatttaggcagagatctttatctgaccctcagcttccctctgtagatatatagatatataaatataaatatgaatataagtatgttttacagcacagcatctgacctgtagatggaggttttgttggttgtttattttcccctcttgcaagtgctacccatgtgagtgtgtgaagtttctctactaagtaaaacacaggcccttttccttgtttgctttgtgttagcttattgtaaacagccatttgttgtaaattattattggcattaaattataatttatgattttcaaagcaaaa

SEQ ID NO: 9 - protein, 414 aa, Homo sapiens
MAMVVSTWRDPQDEVPGSQGSQASQAPPVPGPPPGAPHTPQTPGQGGPASTPAQTAAGGQGGPGGPGSDKQQQQQHIECVVCGDKSSGKHYGQFTCEGCKSFFKRSVRRNLSYTCRANRNCPIDQHHRNQCQYCRLKKCLKVGMRREAVQRGRMPPTQPTHGQFALTNGDPLNCHSYLSGYISLLLRAEPYPTSRFGSQCMQPNNIMGIENICELAARMLFSAVEWARNIPFFPDLQITDQVALLRLTWSELFVLNAAQCSMPLHVAPLLAAAGLHASPMSADRVVAFMDHIRIFQEQVEKLKALHVDSAEYSCLKAIVLFTSDACGLSDVAHVESLQEKSQCALEEYVRSQYPNQPTRFGKLLLRLPSLRTVSSSVIEQLFFVRLVGKTPIETLIRDMLLSGSSFNWPYMAIQ

SEQ ID NO: 10 - protein, 281 aa, Homo sapiens
MQAVWDLEQGKYGFAVQRGRMPPTQPTHGQFALTNGDPLNCHSYLSGYISLLLRAEPYPTSRFGSQCMQPNNIMGIENICELAARMLFSAVEWARNIPFFPDLQITDQVALLRLTWSELFVLNAAQCSMPLHVAPLLAAAGLHASPMSADRVVAFMDHIRIFQEQVEKLKALHVDSAEYSCLKAIVLFTSDACGLSDVAHVESLQEKSQCALEEYVRSQYPNQPTRFGKLLLRLPSLRTVSSSVIEQLFFVRLVGKTPIETLIRDMLLSGSSFNWPYMAIQ

SEQ ID NO: 11 - protein, 261 aa, Homo sapiens
MPPTQPTHGQFALTNGDPLNCHSYLSGYISLLLRAEPYPTSRFGSQCMQPNNIMGIENICELAARMLFSAVEWARNIPFFPDLQITDQVALLRLTWSELFVLNAAQCSMPLHVAPLLAAAGLHASPMSADRVVAFMDHIRIFQEQVEKLKALHVDSAEYSCLKAIVLFTSDACGLSDVAHVESLQEKSQCALEEYVRSQYPNQPTRFGKLLLRLPSLRTVSSSVIEQLFFVRLVGKTPIETLIRDMLLSGSSFNWPYMAIQ

SEQ ID NO: 12 - protein, 261 aa, Homo sapiens; identical to SEQ ID NO: 11

SEQ ID NO: 13 - protein, 414 aa, Mus musculus; identical to SEQ ID NO: 9

SEQ ID NO: 14 - protein, 271 aa, Mus musculus
MGLTAVQRGRMPPTQPTHGQFALTNGDPLNCHSYLSGYISLLLRAEPYPTSRFGSQCMQPNNIMGIENICELAARMLFSAVEWARNIPFFPDLQITDQVALLRLTWSELFVLNAAQCSMPLHVAPLLAAAGLHASPMSADRVVAFMDHIRIFQEQVEKLKALHVDSAEYSCLKAIVLFTSDACGLSDVAHVESLQEKSQCALEEYVRSQYPNQPTRFGKLLLRLPSLRTVSSSVIEQLFFVRLVGKTPIETLIRDMLLSGSSFNWPYMAIQ

SEQ ID NO: 15 - protein, 281 aa, Mus musculus; identical to SEQ ID NO: 10

SEQ ID NO: 16 - protein, 261 aa, Mus musculus; identical to SEQ ID NO: 11

SEQ ID NO: 17 - RNA, 19 nt, artificial
  synthesized
gccgucucaagaagugcuu

SEQ ID NO: 18 - RNA, 19 nt, artificial
  synthesized
cauugagacacugaucaga

SEQ ID NO: 19 - RNA, 19 nt, artificial
  synthesized
gcaagcauuacggugucuu

SEQ ID NO: 20 - RNA, 19 nt, artificial
  synthesized
ccccuagcaugaacuugug

SEQ ID NO: 21 - DNA, 20 bp, artificial
  synthesized
tggtcgcctttatggaccac

SEQ ID NO: 22 - DNA, 20 bp, artificial
  synthesized
gcgaagcaaaagctttccga

SEQ ID NO: 23 - DNA, 20 bp, artificial
  synthesized
ggagcgagctgtttgtgttg

SEQ ID NO: 24 - DNA, 20 bp, artificial
  synthesized
tggtccataaaggcgaccac

SEQ ID NO: 25 - DNA, 20 bp, artificial
  synthesized
tcggaaagcttttgcttcgc

SEQ ID NO: 26 - DNA, 20 bp, artificial
  synthesized
ggccagttaaaactgctgcc

SEQ ID NO: 27 - DNA, 20 bp, artificial
  synthesized
ggcctccaaggagtaagacc

SEQ ID NO: 28 - DNA, 20 bp, artificial
  synthesized
aggggtctacatggcaactg

SEQ ID NO: 29 - DNA, 20 bp, artificial
  synthesized
aaacccccatcgaaaccctc

SEQ ID NO: 30 - DNA, 20 bp, artificial
  synthesized
agtagcaggttgttctgccc

SEQ ID NO: 31 - DNA, 20 bp, artificial
  synthesized
cagggtgtgctgatttggga

SEQ ID NO: 32 - DNA, 20 bp, artificial
  synthesized
gttcccagcagtgagctctt

SEQ ID NO: 33 - DNA, 20 bp, artificial
  synthesized
gcagaggactgtccaagcaa

SEQ ID NO: 34 - DNA, 20 bp, artificial
  synthesized
cctctcaacagccacgctaa

SEQ ID NO: 35 - DNA, 20 bp, artificial
  synthesized
gccatcagagtcaccaatcc

SEQ ID NO: 36 - DNA, 20 bp, artificial
  synthesized
aaacatgcacacaagccatc

(19) United States
(12) Patent Application Publication
(10) Pub. No.: US 2015/0283164 A1
(43) Pub. Date: Oct. 8, 2015

(54) Treatment of Myelodysplastic Syndrome by Inhibition of NR2F2

(71) Applicant: Christine Victoria Ichim, Spring Valley, CA (US)

(72) Inventor: Christine Victoria Ichim, Spring Valley, CA (US)

(21) Appl. No.: 14/588,374

(22) Filed: Dec. 31, 2014

Related U.S. Application Data
(63) Continuation-in-part of application No. 13/652,395, filed on Oct. 15, 2012, now Pat. No. 9,091,696.
Application No. 13/652,395 is a continuation of application No. 12/619,290, filed on Nov. 16, 2009, now abandoned.
(60) Provisional application No. 61/114,764, filed on Nov. 14, 2008.

(51) Int. Cl. A61K 31/713; A61K 45/06; C12N 5/078; A61K 9/127

(52) U.S. Cl. CPC A61K 31/713; A61K 9/127; A61K 45/06; C12N 5/0634; C12N 2501/2311; C12N 2501/33; C12N 2501/145; C12N 2501/22; C12N 2501/998; C12N 2501/125; C12N 2501/26

(57) ABSTRACT

Methods, compositions, and treatment protocols are provided in the current invention for the treatment of myelodysplastic syndrome (MDS) through the inhibition of NR2F2 gene expression or activity of protein. In one embodiment silencing, or substantial inhibition of NR2F2 expression is achieved through induction of RNA interference in cells associated with development of MDS. Induction of differentiation or stimulation of apoptosis as a result of NR2F2 inhibition may be used to reduce the state of MDS, and/or in other embodiments to inhibit or revert progression to leukemic states.

### CROSS-REFERENCE TO RELATED APPLICATIONS

This application claims priority to and is a continuation-in-part to pending Non-Provisional U.S. application Ser. No. 13/652,395 filed Oct. 15, 2012, which claims priority to Non-Provisional U.S. application Ser. No. 12/619,290, filed Nov. 16, 2009, which claims the benefit under 35 USC §119(e) of U.S. provisional application No. 61/114,764 filed Nov. 14, 2008, each of which is hereby expressly incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Apr. 20, 2015, is named CIP4SequenceListing.txt and is 68 kilobytes in size.

### FIELD OF THE INVENTION

The invention pertains to the use of molecular interventions to treat myelodysplastic syndrome (MDS), more specifically the invention relates to utilization of agents that suppress NR2F2 gene transcription or NR2F2 protein function. More specifically the invention provides means of inducing gene silencing or substantial suppression of gene function for targeting the NR2F2 gene for suppression of MDS

### BACKGROUND

Myelodysplastic syndrome is a bone marrow failure syndrome characterized by peripheral blood cytopenias, apoptosis of bone marrow haematopoietic progenitors, abnormal blood cell morphology (dysplasia) and a marked propensity to progress to acute leukaemia[1]. The central paradox of MDS biology resides in the observation that the MDS clone, which is characterized by reduced numbers of mature progeny and by maturing progenitors that exhibit impaired clonogenicity[2] and a high rate of apoptosis[3], nonetheless comes to dominate the bone marrow at the expense of residual normal haematopoiesis and thereby causes disease. Consideration of MDS as a disease of haematopoietic stem cells suggests a resolution to this paradox: the MDS clone, despite the defects seen in its differentiating progeny, out-competes normal haematopoiesis because of a selective advantage at the stem cell level.

MDS and acute myelogenous leukaemia (AML) are closely related diseases of haematopoietic stem cells (HSCs), characterized by effacement of normal blood cell production and accumulation of neoplastic cells, known as blasts, in the bone marrow, peripheral blood, and other tissues. Despite the many advances made in the understanding of the biology of these diseases over the past three decades, therapy for AML and MDS remains, in most cases, debilitating and ineffective, especially in older patients[4].

Allogeneic stem cell transplantation is the only potentially curative therapy for MDS[5], but is feasible only for a small minority of patients owing to age-related comorbidities. A minority of patients respond to treatment with erythropoietin[6] or immunosuppressive therapy[7]. Lenalidomide has shown remarkable efficacy and durable responses in patients with the del(5q) chromosome abnormality[6, 8], which is present in 15-20% of MDS cases[9]. The hypomethylating agents 5-azacytidine[10] and decitabine[11] have a favourable impact on haematopoiesis, survival, quality of life, and progression to acute leukaemia, but responses are seen in only ˜20% of patients, are of limited duration, and are accompanied by significant side effects.

In summary, therapeutic options for patients with AML and MDS are limited. In order to improve efficacy and reduce toxicity of MDS treatment, new therapies must be devised

### SUMMARY OF THE INVENTION

The invention discloses the critical role of NR2F2 in the initiation and maintenance of MDS, as well as progression of MDS to leukemias, such as acute myeloid leukemia (AML). In one aspect of the invention suppression of MDS in part through differentiation is demonstrated to occur subsequent to suppression of the NR2F2 gene by means of induction of RNA interference.

### BRIEF DESCRIPTION OF THE DRAWINGS

It will be appreciated that the drawings are not necessarily to scale, with emphasis instead being placed on illustrating the various aspects and features of embodiments of the invention, in which:

FIG. 1 shows proof of concept for long term culture of a primary MDS patient samples. A cytospin from bone marrow from a patient with myelodysplastic syndrome that was grown in in vitro culture for more than three months.

FIG. 2 shows proof of concept for long term culture of a primary MDS patient samples. A cytospin from bone marrow from a patient with myelodysplastic syndrome that was grown in in vitro culture for more than three months.

FIG. 3 shows proof of concept for long term culture of a primary MDS patient samples. A cytospin from bone marrow from a patient with myelodysplastic syndrome that was grown in in vitro culture for more than three months.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The invention is based on the discovery that NR2F2 is a fundamental factor in the initiation and maintenance of MDS, as well as progression to a leukemic state. Specifically, the invention is based on the discovery that NR2F2 is found specifically on the MDS stem cell, and that inhibition of NR2F2 in MDS stem cells is associated with differentiation of MDS cells into cells lacking malignant phenotype.

The cancer stem cell (CSC) model proposes that each cancer consists of a small population of cells capable of unlimited growth and self-renewal, known as CSCs, and a much larger population of cells, descendants of the CSCs, that have lost self-renewal capacity and are undergoing terminal differentiation[12]. Evidence supporting this model has been reported for several malignancies including AML[13], brain cancer[14, 15] and breast cancer[16]. The CSC model has important implications for cancer therapy; eradication of CSCs, the cells responsible for maintenance of the neoplasm, would be necessary and sufficient to achieve cure. In one aspect of the invention the concept of CSC is applied to MDS, with the invention involving the utilization of NR2F2 silencing as a means of targeting these cells.

The central paradox of MDS biology resides in the observation that the MDS clone, which is characterized by reduced numbers of mature progeny and by maturing progenitors that exhibit impaired clonogenicity[2] and a high rate of apoptosis[3], nonetheless comes to dominate the bone marrow at the expense of residual normal haematopoiesis and thereby causes disease. The CSC model suggests a resolution to this paradox, namely that the MDS clone, despite the defects seen in its differentiating progeny out-competes normal haematopoiesis because of a selective advantage at the stem cell level. As part of the current invention we target the self-renewal properties of MDS stem cells by silencing NR2F2. Determination of genes that regulate self-renewal in cancer has been hindered by the inability to isolate pure populations of self-renewing (clonogenic) cancer cells. Numerous attempts have been made to isolate pure populations of clonogenic cells by fluorescence activated cell sorting based on cellular immunophenotype. While these experiments successfully enrich for human leukaemia cells with clonal longevity, they fail to isolate pure clonogenic cells[13, 17, 18], i.e. even in the “purified” population clonogenic cells are far outnumbered by contaminating non-clonogenic cells, precluding genetic analysis. Therefore characterization of the transcriptome of clonogenic MDS cells has awaited the development of techniques and approaches that permit the study of homogenous populations of clonogenic versus non-clonogenic cells. We identified NR2F2 as being a fundamental factor in MDS self renewal.

The term “NR2F2” as used herein refers to nuclear receptor subfamily2, group F, member 2 and is also referred to as Chicken Ovalbumin Upstream Promoter-Transcription Factor 2 or COUP-TF2 and includes, without limitation, the protein encoded by the gene having the sequence as shown in SEQ ID NO:1 (human) or SEQ ID NO: 5 (mouse) or variants thereof (SEQ ID NO: 2, 3 and 4 for human and SEQ ID NO: 6, 7 or 8 for mouse) and the protein having the amino acid sequence as shown in SEQ ID NO: 9 (human) or SEQ ID NO: 13 (mouse) or variants thereof (SEQ ID NO: 10, 11 and 12 for human and SEQ ID NO: 14, 15 or 16 for mouse).

The term “myelodysplastic syndromes” or “MDS” means hematopoietic stem cell disorders characterized by one or more of the following: ineffective blood cell production, progressive cytopenias, risk of progression to acute leukemia or cellular marrow with impaired morphology and maturation (dysmyelopoiesis). The term “myelodysplastic syndromes” or “MDS” unless otherwise noted includes: refractory anemia, refractory anemia with ringed sideroblasts, refractory anemia with excess blasts, refractory anemia with excess blasts in transformation and chronic myelomonocytic leukemia.

The term “a cell” as used herein includes a plurality of cells and refers to all types of cells including hematopoietic and cancer cells. Administering a compound to a cell includes in vivo, ex vivo and in vitro treatment.

The term “stem cell” as used herein refers to a cell that has the ability for self-renewal. Non-cancerous stem cells have the ability to differentiate where they can give rise to specialized cells.

The term “effective amount” as used herein means a quantity sufficient to, when administered to an animal, effect beneficial or desired results, including clinical results, and as such, an “effective amount” depends upon the context in which it is being applied. For example, in the context of inhibiting self-renewal of stem cells, it is the amount of the NR2F2 inhibitor sufficient to achieve such an inhibition as compared to the response obtained without administration of the NR2F2 inhibitor.

The term “oligonucleotide” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “animal” as used herein includes all members of the animal kingdom, preferably mammal. The term “mammal” as used herein is meant to encompass, without limitation, humans, domestic animals such as dogs, cats, horses, cattle, swine, sheep, goats, and the like, as well as wild animals. In an embodiment, the mammal is human.

The term “interfering RNA” or “RNAi” or “interfering RNA sequence” refers to double-stranded RNA (i.e., duplex RNA) that targets (i.e., silences, reduces, or inhibits) expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes small-interfering RNA″ or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in length). siRNA duplexes may comprise 3′ overhangs of about 1 to about 4 nucleotides, preferably of about 2 to about 3 nucleotides and 5′ phosphate termini. The siRNA can be chemically synthesized or maybe encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al., Nucleic Acids Res. 31: 981-7 (2003); Knight and Bass, Science 293: 2269-71 (2001); and Robertson et al., J. Biol. Chem. 243: 82 (1968)). Preferably, dsRNA are at least 50 nucleotides to about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

The term “antisense nucleic acid” as used herein means a nucleotide sequence that is complementary to its target e.g. a NR2F2 transcription product. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

As used in this context, to “treat” means to ameliorate at least one symptom of the disorder. In some embodiments, a treatment can result in a reduction in tumor size or number, or a reduction in tumor growth or growth rate.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and origin.

One embodiment of the invention is a short-interfering ribonucleic acid (siRNA) molecule effective at silencing NR2F2 expression or substantially inhibiting NR2F2 expression. In one embodiment of the invention the oligonucleotide backbone is chemically modified to increase the deliverability of the interfering ribonucleic acid molecule. In another embodiment these chemical modifications act to neutralize the negative charge of the interfering ribonucleic acid molecule. One embodiment of the invention consists of a pharmaceutical composition comprising an siRNA oligonucleotide that induces RNA interference against NR2F2. It is known to one of skill in the art that siRNAs induce a sequence-specific reduction in expression of a gene by the process of RNAi, as previously mentioned. Thus, siRNA is the intermediate effector molecule of the RNAi process that is normally induced by double stranded viral infections, with the longer double stranded RNA being cleaved by naturally occurring enzymes such as DICER. Some nucleic acid molecules or constructs provided herein include double stranded RNA molecules comprising 16-30, e.g., 16, 17, 18, 19, 20, 21, 22, 23, 24, 25, 26, 27, 28, 29, or 30 nucleotides in each strand, wherein one of the strands is substantially identical, for example at least 85% (or more, as for example, 90%, 95%, or 100%) identical, e.g., having 3, 2, 1, or 0 mismatched nucleotide(s), to a target region in the mRNA of NR2F2 and the other strand is identical or substantially identical to the first strand. However, it will be appreciated that the dsRNA molecules may have any number of nucleotides in each strand which allows them to reduce the level of NR2F2 protein, or the level of a nucleic acid encoding NR2F2. The dsRNA molecules provided herein can be chemically synthesized, or can be transcribed in vitro from a DNA template, or in vivo from, e.g., shRNA, which is mentioned below. The dsRNA molecules can be designed using any method known in the art.

In one embodiment, nucleic acids provided herein can include both unmodified siRNAs and modified siRNAs as known in the art. For example, in some embodiments, siRNA derivatives can include siRNA having two complementary strands of nucleic acid, such that the two strands are crosslinked. For a specific example, a 3′ OH terminus of one of the strands can be modified, or the two strands can be crosslinked and modified at the 3′ OH terminus. The siRNA derivative can contain a single crosslink (one example of a useful crosslink is a psoralen crosslink). In some embodiments, the siRNA derivative has at its 3′ terminus a biotin molecule (for example, a photocleavable molecule such as biotin), a peptide (as an example an HIV Tat peptide), a nanoparticle, a peptidomimetic, organic compounds, or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acids described within the practice of the current invention can include nucleic acids that are unconjugated or can be conjugated to another moiety, such as a nanoparticle, to enhance a desired property of the pharmaceutical composition. Properties useful in the development of a therapeutic agent include: a) absorption; b) efficacy; c) bioavailability; and d) half life in blood or in vivo. RNAi is believed to progress via at least one single stranded RNA intermediate, the skilled artisan will appreciate that single stranded-siRNAs (e.g., the antisense strand of a ds-siRNA) can also be designed as described herein and utilized according to the claimed methodologies.

In one embodiment the pharmaceutical composition comprises a nucleic acid-lipid particle that contains an siRNA oligonucleotide that induces RNA interference against NR2F2. In some aspects the lipid portion of the particle comprises a cationic lipid and a non-cationic lipid. In some aspects the nucleic acid-lipid particle further comprises a conjugated lipid that prevents aggregation of the particles and/or a sterol (e.g., cholesterol).

For practice of the invention, methods for expressing siRNA duplexes within cells from recombinant DNA constructs to allow longer-term target gene suppression in cells are known in the art, including mammalian Pol III promoter systems (e.g., H1 or U6/snRNA promoter systems) capable of expressing functional double-stranded siRNAs. Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression. Constructs containing siRNA sequence(s) under the control of a T7 promoter also make functional siRNAs when co-transfected into the cells with a vector expressing T7 RNA polymerase. A single construct may contain multiple sequences coding for siRNAs, such as multiple regions of the NR2F2 gene, such as a nucleic acid encoding the NR2F2 mRNA, and can be driven, for example, by separate Pol III promoter sites. In some situations it will be preferable to induce expression of the hairpin siRNA or shRNAs in a tissue specific manner in order to activate the shRNA transcription that would subsequently silence NR2F2 expression. Tissue specificity may be obtained by the use of regulatory sequences of DNA that are activated only in the desired tissue. Regulatory sequences include promoters, enhancers and other expression control elements such as polyadenylation signals. Regulatory sequences include those which direct constitutive expression of a nucleotide sequence in many types of host cell and those which direct expression of the nucleotide sequence only in certain host cells. Tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters as follows may be used to target gene expression in other tissues. Examples of more tissue specific promoters include in (a) to target the pancreas promoters for the following may be used: insulin, elastin, amylase, pdr-I, pdx-I, glucokinase; (b) to target the liver promoters for the following may be used: albumin PEPCK, HBV enhancer, a fetoprotein, apolipoprotein C, .alpha.-I antitrypsin, vitellogenin, NF-AB, Transthyretin; (c) to target the skeletal muscle promoters for the following may be used: myosin H chain, muscle creatine kinase, dystrophin, calpain p94, skeletal .alpha.-actin, fast troponin 1; (d) to target the skin promoters for the following may be used: keratin K6, keratin KI; (e) lung: CFTR, human cytokeratin IS (K 18), pulmonary surfactant proteins A, B and C, CC-10, Pi; (0 smooth muscle: sm22 .alpha., SM-.alpha.-actin; (g) to target the endothelium promoters for the following may be used: endothelin-I, E-selectin, von Willebrand factor, TIE, KDR/flk-I; (h) to target melanocytes the tyrosinase promoter may be used; (i) to target the mammary gland promoters for the following may be used: MMTV, and whey acidic protein (WAP).

Yet another embodiment of the invention consists of a pharmaceutical composition comprising an oligonucleotide that induces RNA interference against NR2F2 combined with a delivery agent such as a liposome. For more targeted delivery immunoliposomes, or liposomes containing an agent inducing selective binding to neoplastic cells may be used.

The present invention further provides pharmaceutical compositions comprising the nucleic acid-lipid particles described herein and a pharmaceutically acceptable carrier.

Another embodiment of the invention consists of a pharmaceutical composition comprising an oligonucleotide that induces RNA interference against NR2F2 combined with an additional chemotherapeutic agent.

Yet another embodiment of the invention consists of a pharmaceutical composition comprising an oligonucleotide that induces RNA interference against NR2F2 combined with an additional agent used to induce differentiation.

One embodiment of the invention is a short-interfering ribonucleic acid (siRNA) molecule effective at silencing NR2F2 expression that has been cloned in to an appropriate expression vector giving rise to an shRNA vector.

In certain embodiment shRNA olignucleotides are cloned in to an appropriate mammalian expression vectors, examples of appropriate vectors include but are not limited to lentiviral, retroviral or adenoviral vector.

In this embodiment, the invention consists of a viral vector, comprising the inhibitory RNA molecule described above. The viral vector preferably is a lentivirus. In one aspect the viral vector is capable of infecting cancer cells. Another embodiment is a lentivirus vector that is an integrating vector. The viral vector preferably is capable of transducing cancer cells. The viral vector is preferably packaged in a coat protein the specifically binds to cancer cells. The viral vector preferably is capable of expressing an RNA that inhibits NR2F2 expression. Another embodiment of the invention is one in which the viral vector is preferably produced by a vector transfer cassette and a separate helper plasmid. In certain embodiment the shRNA olignucleotides is combined with a pharmaceutically acceptable vehicle a pharmaceutical composition. One embodiment is a pharmaceutical composition comprising an inhibitory oligonucleotide that is a double stranded RNA molecule.

One aspect of the invention is a microRNA or family of microRNAs are administered that substantially inhibit expression of NR2F2.

The present inventors have found that NR2F2 is a regulator of cancer cell proliferation, self-renewal and differentiation, and that silencing of NR2F2 with oligonucleotides that induce RNA interference induces a reduction of cancer cell proliferation, inhibiting clonogenicity and self-renewal of proliferating cancer cells, and induces differentiation.

Accordingly, the present disclosure provides a method of modulating MDS, proliferation and/or differentiation comprising administering an effective amount of a synthetic oligonucleotide that induces RNA interference of NR2F2 to a cell or animal in need thereof.

In one aspect, the synthetic oligonucleotide is an siRNA targetting NR2F2. In another aspect, the synthetic oligonucleotide is an shRNA targeting NR2F2. And yet in another aspect the synthetic oligonucleotide is an antisense RNA molecule targeting NR2F2.

Accordingly, the present disclosure provides a method of inhibiting self-renewal of stem cells comprising administering an effective amount of an oligonucleotides that induce RNA interference to a cell or animal in need thereof. The present disclosure also provides the use of a oligonucleotides that induce RNA interference for inhibiting self-renewal of stem cells in a cell or animal in need thereof. The present disclosure further provides the use of an oligonucleotide that induce RNA interference in the preparation of a medicament for inhibiting self-renewal of stem cells in a cell or animal in need thereof. The present disclosure also provides a oligonucleotides that induce RNA interference for use in inhibiting self-renewal of stem cells in a cell or animal in need thereof.

In another embodiment, the present disclosure provides a method of inducing terminal differentiation of stem cells comprising administering of an effective amount of oligonucleotides that induce RNA interference to NR2F2 to a cell or animal in need thereof. The present disclosure also provides the use of oligonucleotides that induce RNA interference to NR2F2 for inducing terminal differentiation of stem cells in a cell or animal in need thereof. The present disclosure further provides the use of oligonucleotides that induce RNA interference to NR2F2 in the preparation of a medicament for inducing terminal differentiation of stem cells in a cell or animal in need thereof. The present disclosure also provides oligonucleotides that induce RNA interference to NR2F2 for use in inducing terminal differentiation of stem cells in a cell or animal in need thereof.

### EXAMPLES

### Materials and Methods

### Cell Lines

U937 and 32Dc13 cells were purchased from ATCC (Manassas, Va.). The 293GPG retroviral packaging cell line was a gift of Richard Mulligan, Harvard University. U937 cells were purchased from ATCC and grown in RPMI supplemented with 10% FBS. 32Dc13 cells were purchased from ATCC and grown in RPMI with 1 ng/mL of rmIL-3. The 293GPG retroviral packaging cell line (a gift of Richard Mulligan, Harvard University) was grown in DMEM medium supplemented with 10% FBS, tetracycline (1 mg/mL), G418 (0.3 mg/mL) and puromycin (2 mg/mL).

### Culture of Primary Human Myelodysplastic Syndrome Cells

Fresh primary human myelodysplastic syndrome cells were attained with informed consent following institutional REB approval. Progenitor cells and blasts were enriched by negative selection using RosetteSep concurrent with Ficoll-Paque gradient centrifugation. Mononuclear cells were cultured on a monolayer of irradiated OP9 cells in IMDM medium supplemented with 30 ng/mL TPO, 30 ng/mL rhIL-11, 30 ng/mL rhFlt-3 ligand, 50 ng/mL rhG-CSF, 30 ng/mL rhGM-CSF, 30 ng/mL rh SCF, 10 ug/mL insulin, 5 ug/mL tranferrin, 0.5% BSA Fraction V, 5% FBS; herein referred to as MDS cell medium. Finally, cultures were maintained in a nitrogen incubator under hypoxic conditions (2% oxygen).

Generation of shRNA—

Oligonucleotides targeting human or mouse NR2F2 were synthesized (Sigma-Genosys, Oakville, ON Canada), annealed and cloned into the pSiren vector (Clonetech, Mountain View Calif.), after which sequence was verified at The Centre for Applied Genomics (TCAG), Toronto, ON Canada. Virus was prepared by transient transfection of plasmid in the 293GPG cell line as described above.

Generating shRNA Retrovirus—

The 293GPG retroviral packaging cell line (a gift of Richard Mulligan, Harvard University) was grown in DMEM medium supplemented with 10% FBS, tetracycline (1 mg/mL), G418 (0.3 mg/mL) and puromycin (2 mg/mL). VSV-G pseudotyped retroviral particles were generated by transient transfection of 293GPG cells. 293GPG cells were cultured in 15 cm plates with 30 mL of 293GPG medium. 12 hours after removal of antibiotics, cells were transiently transfected with 25 μg of plasmid DNA using Lipofectamine 2000 (Invitrogen). Virus was collected on days 3 to 7, concentrated by centrifugation at 16,500 RPM for 90 minutes. Transduction of >95% of cells was confirmed by flow cytometry

Generation of shRNA Lentivirus—

The packaging vectors pRSV Rev, pMD2.G (VSV-G) and pMDLg/pRRE, as well as the shRNA vector H1GIP (a kind gift from John Dick, University Health Network) were grown in STBL2 competent cells (Invitrogen, Carlsbad, Calif.) at 30 degrees. Plasmid DNA was extracted using the EndoFree Mega kit (Qiagen).

293T/17 cells were passaged 1:4 to 1:6 three times a week, before reaching 80% confluence. This passaging schedule was intended to maintain the cells at a density where they would be in a log state of proliferation, as well as to maintain them as individual cells (as opposed to cell aggregates) which would also increase transfection efficiency. Only early passages of the 293T/17 cells lines were used for the production of lentivirus, furthermore, batches of cells were not maintained in culture for more than a month. Care was taken to maintain 293T/17 cells endotoxin free.

293T/17 cells were transfected using the CalPhos Mammalian Transfection Kit (Clonetech, Palo Alto, Calif.) in 15 cm plates. Briefly, 12×106 cells were plated in a 15 cm dish the day prior to transfection. Two hours before transfection medium was aspirated and cells were fed 25 mL of fresh medium. Calcium Phosphate precipitates were prepared in 50 mL conical tubes in master mixes sufficient for transfecting 6 plates. Each plate received a solution containing 63.4 μg of DNA (28.26 μg of the H1 shRNA hairpin vector; 18.3 μg of pMDLg/pRRE; 9.86 μg of pMD2.G and 7.04 μg of pRSV Rev) and 229.4 μL of 2 M Calcium solution in a total volume of 3.7 mL. The transfection solution was incubated 20 minutes at room temperature and was then added drop wise to each plate. Plates were incubated overnight with transfection precipitate, and washed with PBS the next morning.

Lentiviral supernatent was collected after 24 and 48 hours. Supernatant was centrifuged in a table-top centrifuge for 10 minutes to remove debris and then pooled and filtered through a 0.45 μm pore size polyethersulfone (PES) bottle-top filter (Nalgene, Thermo Fisher Scientific). Ultracentrifugation was conducted as described above.

Transduction of Bone Marrow Cells—

12-week old C57B1/6 mice were given 5 fluorouracil, 150 μg/g body mass, by intraperitoneal injection and humanely killed ninety-six hours later. Bone marrow was collected from femurs and tibiae and cultured in Iscove's Modified Dulbecco's Medium previously conditioned by culturing on OP-9 cells (T Nakano, Japan) for 72 hours, supplemented with fetal bovine serum (5%), c-Kit ligand conditioned medium (3%), Flt-3 ligand (30 ng/mL), TPO (30 ng/mL), IL-11 (30 ng/mL), Insulin (10 μg/mL), bovine serum albumin (0.5%), conditions that minimize differentiation but initiate cycling of long-term repopulating cells.

Following prestimulation, 2.0×106 cells were seeded per well of a 24 well plate in 400 μL of bone marrow culture medium, plus 4 μg/mL polybrene (Sigma) and 10 mM HEPES (Gibco-Invitrogen). 75-150 μL of retrovirus was added to the cells to give an MOI of what our method of titration estimated to be 100. One round of spin-infection was carried out by centrifugation at 3000 RPM on a Beckman GH 3.8 rotor for 45 minutes at room temperature. Forty-eight hours after retroviral transduction GFP-positive cells were assessed by flow cytometry.

Immunoblotting—

Immunoblotting for human NR2F2 was performed using the PP-N2025-00 (Perseus Proteomics, Tokyo, Japan), or ab12982 (Abcam, Cambridge, Mass.) antibodies, while immunoblotting for mouse NR2F2 was performed using the LS-C40527 (LifeSpan Biosciences, Seattle, Wash.) antibody. Western blot analysis. Cells were lysed in RIPA lysis buffer (1% SDS, 1% Triton X-100, 1% deoxycholic acid) and quantified using the DC Protein Assay kit (Bio-Rad). Proteins (25-50 μg) in lysates were resolved on 10% SDS-PAGE gels and transferred to nitrocellulose membrane (Protran, Whatman). The membranes were blocked with 5% non-fat dry milk in 0.1% TBS/Tween-20 or 2% BSA-TBS/Tween-20 (CD95, CD95L and E-cadherin) and incubated in primary antibodies diluted in blocking solution at 4° C. overnight. After incubation with secondary antibodies, detection was performed using the ECL method (Amersham Pharmacia Biotech) and developed using a chemiluminescence imager, G:BOX Chemi XT4 (Synoptics).

Quantitative PCR—

RNA was isolated from 1×106 cells using Trizol reagent (Invitrogen, Burlington, ON Canada) and first strand cDNA was synthesized using SuperScript II Reverse Transcriptase (Invitrogen) according to manufacturer's instructions. Real time PCR was performed according to manufacturer's instructions using SYBR Green Master Mix (Applied Biosystems, Foster City, Calif.) and analyzed using the delta-delta CT method. Primer sequences selected to amplify all transcript variants of NR2F2 and are as follows:

| Human NR2F2 pair1: | |
|---|---|
| SEQ ID NO: 21 | |
| Fwd: | TGGTCGCCTTTATGGACCAC |
| SEQ ID NO: 22 | |
| Revs: | GCGAAGCAAAAGCTTTCCGA |
| Human NR2F2 pair2: | |
| SEQ ID NO: 23 | |
| Fwd: | 5′-GGAGCGAGCTGTTTGTGTTG-3′ |
| SEQ ID NO: 24 | |
| Revs: | 5′-TGGTCCATAAAGGCGACCAC-3′ |
| Human NR2F2 pair3: | |
| SEQ ID NO: 25 | |
| Fwd: | 5′-TCGGAAAGCTTTTGCTTCGC-3′ |
| SEQ ID NO: 26 | |
| Revs: | 5′-GGCCAGTTAAAACTGCTGCC-3′ |
| Human GAPDH: | |
| SEQ ID NO: 27 | |
| Fwd: | 5′-GGCCTCCAAGGAGTAAGACC-3′ |
| SEQ ID NO: 28 | |
| Revs: | 5′-AGGGGTCTACATGGCAACTG-3′ |
| 3′ end Mus NR2F2 pair 1: | |
| SEQ ID NO: 29 | |
| Fwd: | 5′-AAACCCCCATCGAAACCCTC-3′ |
| SEQ ID NO: 30 | |
| Revs: | 5′-AGTAGCAGGTTGTTCTGCCC-3′ |
| 3′ end Mus NR2F2 pair 2: | |
| SEQ ID NO: 31 | |
| Fwd: | 5′-CAGGGTGTGCTGATTTGGGA-3′ |
| SEQ ID NO: 32 | |
| Revs: | 5′-GTTCCCAGCAGTGAGCTCTT-3′ |
| 3′ end Mus NR2F2 pair 3: | |
| SEQ ID NO: 33 | |
| Fwd: | 5′-GCAGAGGACTGTCCAAGCAA-3′ |
| SEQ ID NO: 34 | |
| Revs: | 5′-CCTCTCAACAGCCACGCTAA-3′ |
| 3′ end Mus L32: | |
| SEQ ID NO: 35 | |
| Fwd: | 5′-GCCATCAGAGTCACCAATCC-3′ |
| SEQ ID NO: 36 | |
| Revs: | 5′-AAACATGCACACAAGCCATC-3′ |

Flow Cytometry—

For analysis of c-kit+, sca-1+, lineage-(KSL) cells, red blood cell depleted bone marrow cells were stained with a cocktail containing biotin CD3, biotin CD45R/B220 (RA3-6B2), biotin CD11b (M1/70), biotin erythroid marker (TER-119), biotin Ly-6G (RB6-8C5), c-kit APC, sca-1 PE-Cy7 and either CD34 PE or CD49b PE (all eBioscience) in the dark. Bone marrow was washed once and incubated with streptavidin PE-Cy5 for 20 minutes in the dark. Bone marrow was washed twice and analyzed using flow cytometry on a Becton Dickinson LSR II. All samples analyzed were gated based on FSC/SSC and GFP+ cells. The population of KSL cells is highly enriched for hematopoietic stem cell activity. This population was analyzed and further subdivided based on the expression of the CD34 and CD49b antigen.

Ex vivo suspension culture—Following transduction of mouse bone marrow with MMP-GFP or MMP-NR2F2, cells were placed unsorted into IMDM with 5% FBS, 10% v/v IL-3 conditioned medium from WEHI-3 cells, 1 ng/mL IL-6 and 3% v/v c-kit ligand conditioned medium. Following ten days of culture the cells were washed twice with PBS, stained with either fluorescently labeled c-kit or with fluorescently labeled CD11b and GR-1, and analyzed by flow cytometry.

Liposomal siRNA and miRNA Preparation:—

For in vivo delivery, siRNA and miRNA were incorporated into liposomal 1,2-dioleoyl-sn-glycero-3-phosphatidylcholine (DOPC). DOPC and siRNA or miRNA were mixed in the presence of excess tertiary butanol at a ratio of 1:10 (w/w) siRNA/DOPC. Before in vivo administration, the preparation was hydrated with normal 0.9% saline (100 μL/mouse) for intravenous or intraperitoneal injection.

In Vivo Treatment with si-NR2F2-DOPC—

NR2F2 siRNA, and control siRNA were purchased from Dharmacon. These siRNAs were conjugated with DOPC as described above. The appropriate dosage for treatment was determined by conducting dose-response analysis. For in vivo combination analysis, female athymic nude mice (NCr-nu) were injected into the peritoneal cavity with 1×106 myelodysplastic cells. Mice were divided into two groups (n=12 per group): (i) Control siRNA, and (ii) siNR2F2-DOPC. One week after injection, each siRNA was given twice weekly at 200 μg/kg body weight. Treatment was continued until control mice became moribund, and the last treatment was done 48 or 24 hours before sacrificing them.

siRNA Transfection of Cell Lines with siRNA—

For siRNA transfection, cells grown in 12-well plates were submitted to lipofection using 6 μl of the HiperFect reagent (Qiagen) and 150 ng/well of either negative control siRNA or NR2F2 siRNA. For each experiment at least four siRNA targeting different sequences were used.

Hoechst Side Population:—

To identify and isolate side population (SP) and non-SP fractions, HeyA8 and SKOV3ip1 cells were removed from the culture dish with trypsin and EDTA, pelleted by centrifugation, washed with phosphate-buffered saline (PBS), and resuspended at 37 degree C. in Dulbecco's modified Eagle's medium (DMEM) containing 2% FBS and 1 mM HEPES. The cells were then labeled with Hoechst 33342 (Invitrogen) at a concentration of 5 mg/mL. The labeled cells were incubated for 120 minutes at 37 degree C., either alone or with 50 uM verapamil (Sigma-Aldrich, St. Louis). After staining, the cells were suspended in Hanks' balanced saline solution (HBSS; Invitrogen) containing 2% FBS and 1 mM HEPES, passed a through 40 mm mesh filter, and maintained at 4 degree C. until flow cytometry analysis. The Hoechst dye was excited at 350 nm, and its fluorescence was measured at two wavelengths using a 450 DF10 (450/20 nm band-pass filter) and a 675LP (675 nm long-pass edge filter) optical filter. The gating on forward and side scatter was not stringent, and only debris was excluded.

Proliferation Assay (MTS)—

Cells were seeded in 96-well plates and incubated at 37° C. Cell viability was determined in triplicate at various time points using the MTS assay according to the manufacturer's instructions (Promega). Plates were analysed at OD 490 using an iMark Microplate Reader (Bio-rad). Data are represented as means±s.d.

CFSE Staining—

In all, 500,000 cells were incubated with 10 μM CFSE (Molecular Probes) in PBS for 10 min at 37° C. Cells were washed with 5 volumes of ice-cold PBS and left on ice for 5 min, then washed three times in warm media and either analysed by FACS or replated. Dead cells were excluded by 7AAD staining, which was carried out by adding 5 μl of a 1-mg ml-1 solution of 7AAD to 200 μl of cells and incubated for 30 min at 4° C. in the dark.

Cell Death Assays—

Different cell death assays were used, depending on specific experimental requirements. To quantify DNA fragmentation after a treatment, both dead and live cells were collected for the assay. The total cell pellet was resuspended in 0.1% sodium citrate, pH 7.4, 0.05% Triton X-100 and 50 μg ml-1 propidium iodide. After 2-4 h in the dark at 4° C., fragmented DNA (% subG1 nuclei) was quantified with flow cytometry. To stain cells with DAPI, after a treatment, both dead and live cells were collected and resuspended in 200-300 μl of media, and DAPI was added at 0.025 mg ml-1. Percent dead cells (DAPI-positive) was monitored using FACS in combination with FSC-A and SSC-A gating. To quantify cell death using the trypan blue exclusion assay, cells were resuspended in media and an equal volume of Trypan blue solution (Cellgro) was added. Both living and dead (blue) cells were counted on a haemocytometer under a light microscope.

### Example 1

NR2F2 is highly expressed in both long and short term haematopoietic stem cells and that expression of NR2F2 in bone marrow from patients with acute myelogenous leukemia (AML), chronic myelomonocytic leukemia (CMML) and myelodysplastic syndrome (MDS) is greater compared to control.

### Example 2

When bone marrow overexpressing NR2F2 is transplanted into irradiated mice, the mice develop bone marrow dysplasia, a characteristic of patients with myelodysplastic syndrome. NR2F2 over-expression in vivo causes abnormal localization of immature precursors (ALIP), a clinical characteristic of patients with myelodysplastic syndrome. NR2F2 over-expression inhibits blood cell differentiation and maturation in vivo along the red blood cell lineage. A clinical characteristic of patients with myelodysplastic syndrome is the inability to differentiate their bone marrow cells, giving rise to cytopenias, similar to the ones observed herein. NR2F2 over-expression inhibits blood cell differentiation and maturation in vivo along the red blood cell lineage. A clinical characteristic of patients with myelodysplastic syndrome is the inability to differentiate their bone marrow cells, giving rise to cytopenias, similar to the ones observed herein. NR2F2 over-expression inhibits blood cell differentiation and maturation in vivo along the platelet lineage. A clinical characteristic of patients with myelodysplastic syndrome is the inability to differentiate their bone marrow cells, giving rise to cytopenias, similar to the ones observed herein. NR2F2 over-expression inhibits blood cell differentiation and maturation in vivo along the white blood cell lineage. A clinical characteristic of patients with myelodysplastic syndrome is the inability to differentiate their bone marrow cells, giving rise to cytopenias, similar to the ones observed herein. NR2F2 over-expression inhibits blood cell differentiation and maturation in vivo along the granulocytic lineage. A clinical characteristic of patients with myelodysplastic syndrome is the inability to differentiate their bone marrow cells, giving rise to cytopenias, similar to the ones observed herein.

### Example 3

over-expression of NR2F2 in the bone marrow of healthy animals resulted in a fatal hematological condition that resembles human myelodysplastic syndrome.

### Example 4

Silencing of NR2F2 Induces Differentiation in Malignant and Non-Malignant Hematopoietic Stem cells. Quantification of NR2F2 protein levels, determined by immunoblot and quantified using densitometry, in human 32Dc13 undifferentiated hematopoietic cells that were treated with NR2F2 shRNA or a hairpin control show that knocking down of NR2F2 reduced NR2F2 protein expression. Cytospins demonstrate morphologically that knock down of NR2F2 using short-hairpin RNAs induces terminal differentiation and blood cell maturation of 32Dc13 undifferentiated hematopoietic cells. This demonstrates alleviation of the block in differentiation that causes the cytopenia observed in MDS. Cytospins demonstrate morphologically that knock down of NR2F2 using short-hairpin RNAs induces terminal differentiation and blood cell maturation of 32Dc13 undifferentiated hematopoietic cells. This demonstrates alleviation of the block in differentiation that causes the cytopenia observed in MDS. Dot plots generated by flow cytometry show that knock down of NR2F2 using short-hairpin RNAs induces terminal differentiation and blood cell maturation of 32Dc13 undifferentiated hematopoietic cells. These data demonstrate that knockdown of NR2F2 was sufficient to allow the leukemia cells to become mature granulocytes blood cells hence alleviating the block in differentiation that causes MDS. Knock down of NR2F2 using short-hairpin RNAs induces rapid differentiation of immature bone marrow cells. Bone marrow was grown in suspension culture for several days. While scrambled control shRNA treated cells retained a population of immature bone marrow cells, cultures treated with shRNA against NR2F2 caused differentiation of the immature bone marrow cells along the granulocytic lineage. Knock down of NR2F2 using short-hairpin RNAs increases the differentiation of immature bone marrow cells giving rise to larger bone marrow colonies in methylcellulose culture. This is evident in both multilineage colonies, but especially in erythroid colonies that gave rise to red blood cell precursors.

### Example 5

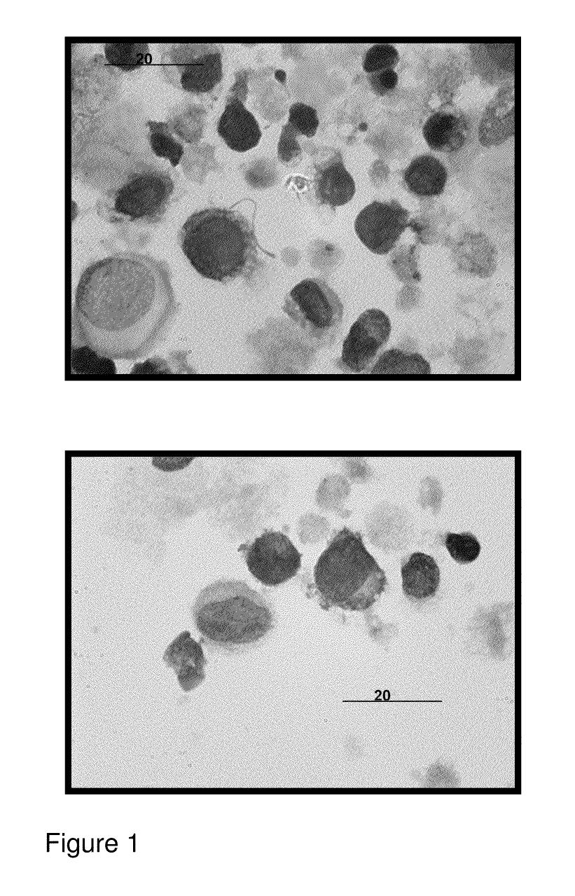

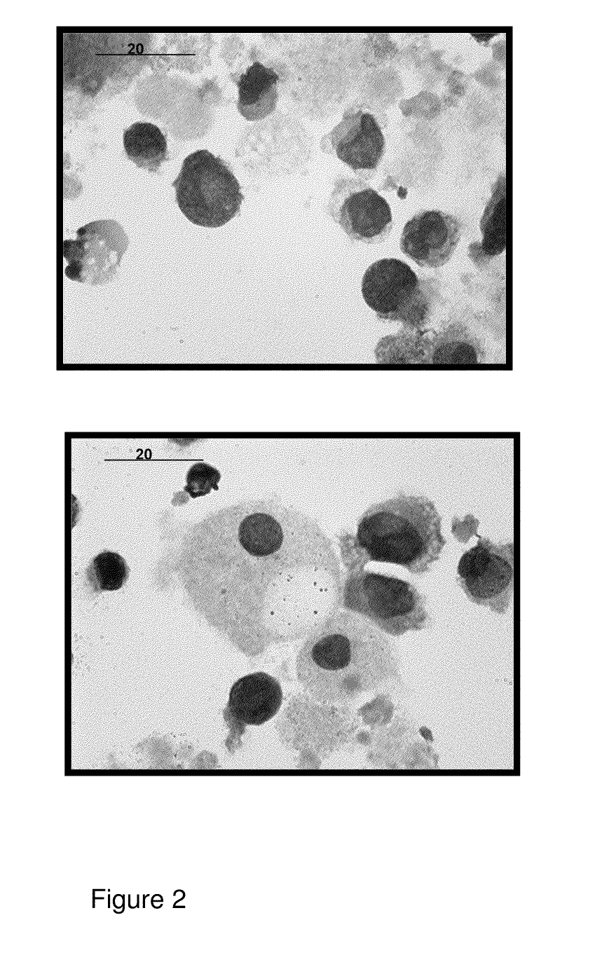

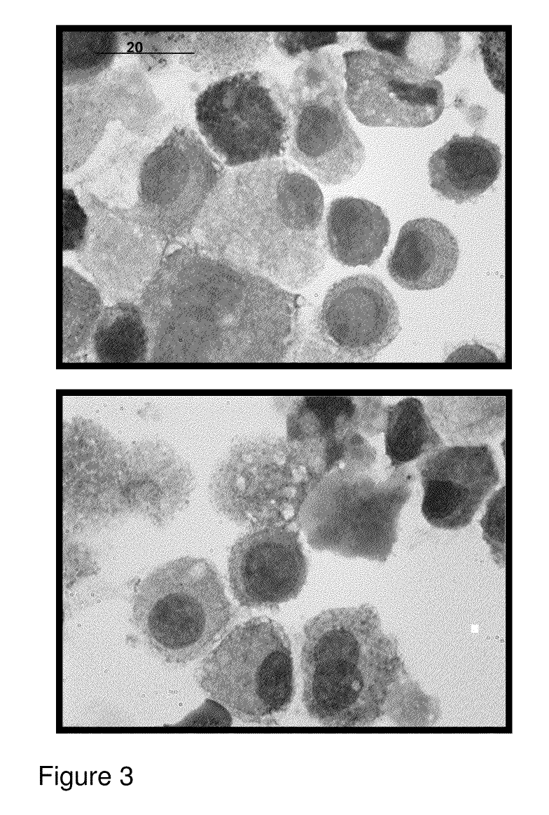

Silencing of EAR-2 induces differentiation in human MDS cells. The question of whether EAR-2 knockdown is able to induce differentiation of human myelodysplastic syndrome samples was assessed. To test this question, we first devised a method of culturing primary MDS patient samples. While primary cells are difficult to culture the following are optimized conditions that have allowed for the maintenance of MDS cells in culture for a period of greater than 6 months. FIGS. 1, 2 and 3 show cytospins of cells from an MDS patient that had been maintained for 3 and 4 months in culture (growing at an exponential rate), respectively, using the conditions proposed here.

Progenitor cells and blasts were enriched by negative selection using RosetteSep concurrent with Ficoll-Paque gradient centrifugation. Mononuclear cells were cultured on a monolayer of irradiated OP9 cells in IMDM medium supplemented with 30 ng/mL TPO, 30 ng/mL rhIL-11, 30 ng/mL rhFlt-3 ligand, 50 ng/mL rhG-CSF, 30 ng/mL rhGM-CSF, 30 ng/mL rh SCF, 10 ug/mL insulin, 5 ug/mL tranferrin, 0.5% BSA Fraction V, 5% FBS; herein referred to as MDS cell medium. Finally, cultures were maintained in a nitrogen incubator under hypoxic conditions (2% oxygen). We found that it was imperative to use fresh MDS patient samples, as viably frozen cells did not grow very well.

Patient samples were either transfected with siRNA to NR2F2 or transduced with shRNA to NR2F2, or the scrambled control respectively. Cells were cultured and assessed for maturation twice a week until terminal differentiation occurred. Flow cytometry was used to look for maintenance of the primitive phenotype based on expression of CD34, CD38, and CD117. Maturation was assessed by expression of CD41 and CD61 for megakaryocyte; glycophorin A+, CD45− for erythrocytes; CD33+, CD45+ and CD14+, CD45+ for myeloid differentiation. Samples treated with NR2F2 siRNA had fewer immature cells, and expressed markers associated with maturation.

Flow cytometry was complemented by observing for morphological differentiation on cytospins stained with May-Grunwald Giemsa. Treatment of MDS patient cultures with siRNA to NR2F2 induced morphological differentiation. Differentiation was confirmed by nitroblue tetrazolium staining for myeloid differentiation and with benzidine staining for erythroid differentiation. Treatment of MDS patient cultures with siRNA to NR2F2 but not scrambled control induced positive staining for differentiation with nitroblue tetrazolium or benzidine. We observed induction of terminal differentiation followed by a wave of massive cell death. Cell growth rates were monitored at time of passage by trypan blue exclusion and apoptosis was assessed bi-weekly by flow cytometry for annexin V-PE and 7AAD and active caspase-3-PE. The ability to differentiate was measured by culture in Methocult H4435, methycellulose medium that favours multilineage differentiation of enriched populations.

### REFERENCES

1. Disperati, P., et al., Progression of myelodysplasia to acute lymphoblastic leukaemia: Implications for disease biology. Leuk Res, 2006. 30(2): p. 233-9.
2. Michalopoulou, S., et al., Impaired clonogenic growth of myelodysplastic bone marrow progenitors in vitro is irrelevant to their apoptotic state. Leuk Res, 2004. 28(8): p. 805-12.
3. Parker, J. E. and G. J. Mufti, The myelodysplastic syndromes: a matter of life or death. Acta Haematol, 2004. 111(1-2): p. 78-99.
4. Wells, R. A., Blood, in The Macmillan Encyclopedia of Aging, D. J. Ekert, Editor. 2002, Macmillan Reference USA: New York.
5. Runde, V., et al., Bone marrow transplantation from HLA-identical siblings as first-line treatment in patients with myelodysplastic syndromes: early transplantation is associated with improved outcome. Chronic Leukemia Working Party of the European Group for Blood and Marrow Transplantation. Bone Marrow Transplant, 1998. 21(3): p. 255-61.
6. List, A., et al., Lenalidomide in the myelodysplastic syndrome with chromosome 5q deletion. N Engl J Med, 2006. 355(14): p. 1456-65.
7. Lim, Z. Y., et al., Low IPSS score and bone marrow hypocellularity in MDS patients predict hematological responses to antithymocyte globulin. Leukemia, 2007. 21(7): p. 1436-41.
8. List, A. F., et al., Efficacy and Safety of CC5013 for Treatment of Anemia in Patients with Myelodysplastic Syndromes (MDS). Blood, 2003. 102(11).
9. Mufti, G. J., Pathobiology, classification, and diagnosis of myelodysplastic syndrome. Best Pract Res Clin Haematol, 2004. 17(4): p. 543-57.
10. Silverman, L. R., et al., Randomized controlled trial of azacitidine in patients with the myelodysplastic syndrome: a study of the cancer and leukemia group B. J Clin Oncol, 2002. 20(10): p. 2429-40.
11. Kantarjian, H., et al., Results of a randomized study of 3 schedules of low-dose decitabine in higher-risk myelodysplastic syndrome and chronic myelomonocytic leukemia. Blood, 2007. 109(1): p. 52-7.
12. Ichim, C. V. and R. A. Wells, First among equals: The cancer cell hierarchy. Leukemia and Lymphoma (in press), 2006.
13. Bonnet, D. and J. E. Dick, Human acute myeloid leukemia is organized as a hierarchy that originates from a primitive hematopoietic cell. Nat Med, 1997. 3(7): p. 730-7.
14. Singh, S. K., et al., Identification of a cancer stem cell in human brain tumors. Cancer Res, 2003. 63(18): p. 5821-8.
15. Singh, S. K., et al., Identification of human brain tumour initiating cells. Nature, 2004. 432(7015): p. 396-401.
16. Al-Hajj, M., et al., Prospective identification of tumorigenic breast cancer cells. Proc Natl Acad Sci USA, 2003. 100(7): p. 3983-8.
17. Lapidot, T., et al., A cell initiating human acute myeloid leukaemia after transplantation into SCID mice. Nature, 1994. 367(6464): p. 645-8.
18. Blair, A., D. E. Hogge, and H. J. Sutherland, Most acute myeloid leukemia progenitor cells with long-term proliferative ability in vitro and in vivo have the phenotype CD34(+)/CD71(−)/HLA-DR. Blood, 1998. 92(11): p. 4325-35.

All references listed herein are expressly incorporated by reference in their entireties. The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

### SEQUENCE LISTINGS

SEQ ID NO: 1
Homo sapiens nuclear receptor subfamily 2,
group F, member 2 (NR2F2), transcript
variant 1, mRNA
NCBI Reference Sequence: NM_021005.3
>gi|223555947|ref|NM_021005.3| Homo sapiens
nuclear receptor subfamily 2, group F,
member 2 (NR2F2), transcript variant 1, mRNA
GCCGTACTGCCTTTTTTCCCCTCTTTCATTCTTTCTCTCCGTCTTTTTCT
CCCCCCTCTGCGCACGAAGGATGTGCTTCTAGGTGGTGATCTGCCCTCC
TCTCTCTCTTTTATCATTTCTCCCCCGCCGCCGGCGAGTTGACTCTTTCC
CTATGTGTGTGAGGCGGCGGCGGCAGCAGCAGCAGCAGCGGCTCCGG
CGGCGGCAGCAGCGGCAGCAGCGACTTCAGCGGCGGCGGCGGCGCTA
GACGCAGCGGCTCCGGGCCCGACCCGGCGGCTTCGGCGGCGGCTCCG
GCGGCAGCGGCGGCCCGGGCGGCCCGCAGGGAACGGCGAGCGGCCTC
CACCCAGCGACTGCGGGCGGCGGCGGCCGGAGAGAGCGAGGCGCGCG
CCGGACGCCCGGGGCAGGCGGCGGCGGCGGCGGCCCAGCGCCAGGAC
GACGCCGCGCAGCGCCCGACGCGGACCACTTTCATGCTGATTCCCCCG
GACCCGGGCAGCGCTCCGGCCACTCCGCGGGCCGCCGGCCTCCGCCCC
GGCCTGCCTGGCTCCCTGGGCGCGCCCGCACCCGGCGCCTCCGATCTC
CTAGTCCTCCTGATTTCGATGGCTTTCCTGAATGGCTGACTGTGGGCTG
CCCTGGACTTGGCCCCCGGACAGTCGCCTCTCCTCCTCCTCTACCTCCT
CCTTCACCACCACCTCCTCTTCCTCCTCCTCCTCCTCCTCCTCCTCCGCC
AACTCCTCGGCTGCACACCAGCTCTAAGAGCGAGAGTGAACGAGAGA
GGGAGGGAGAGAGTGAGAGCGAGCGAGATCTTTGGAGAGATTTTTTT
TTTTGCCTCCTACTTCTGTCTTGAAGCCAGACAATCGACTTCAGCTCTC
CCTCCCCTCCCTCTTTCTCCACGTTCTGCTCCCACTCGCTCTCCTGTCCC
CTTCCCCTCCCCTCCCGGCGGAAAGCCCCCCGAAACCAACAAAGCTGA
GCCGAGAGAAACAAACAAAACAAACACACCGGGCCAGACAAGCCAT
CGACAAAACTTTGCAAAAGCAAAAACAAAAAAGGAAAAACTAACCAA
CCTCAACCAACCAGCCCCCGAGCCACCCGGGGCGCCCTCCCGCGCCCT
CTTGCACCCTCGCACACACAAAAGGCGGCGCGCCGGAGCCCGAGACC
CGGGGAGCCGCCGCCGCCCCGCCGCCGCCCGCAGCCAGGGGAGCAGG
AAGTCCGGACGCAGCCCCCATAGATATGGCAATGGTAGTCAGCACGT
GGCGCGACCCCCAGGACGAGGTGCCCGGCTCACAGGGCAGCCAGGCC
TCGCAGGCGCCGCCCGTGCCCGGCCCGCCGCCCGGCGCCCCGCACACG
CCACAGACGCCCGGCCAAGGGGGCCCAGCCAGCACGCCAGCCCAGAC
GGCGGCCGGTGGCCAGGGCGGCCCTGGCGGCCCGGGTAGCGACAAGC
AGCAGCAGCAGCAACACATCGAGTGCGTGGTGTGCGGAGACAAGTCG
AGCGGCAAGCACTACGGCCAGTTCACGTGCGAGGGCTGCAAGAGCTT
CTTCAAGCGCAGCGTGCGGAGGAACCTGAGCTACACGTGCCGCGCCA
ACCGGAACTGTCCCATCGACCAGCACCATCGCAACCAGTGCCAGTACT
GCCGCCTCAAAAAGTGCCTCAAAGTGGGCATGAGACGGGAAGCGGTG
CAGAGGGGCAGGATGCCGCCGACCCAGCCGACCCACGGGCAGTTCGC
GCTGACCAACGGGGATCCCCTCAACTGCCACTCGTACCTGTCCGGATA
TATTTCCCTGCTGTTGCGCGCGGAGCCCTATCCCACGTCGCGCTTCGGC
AGCCAATGCATGCAGCCCAACAACATCATGGGTATCGAGAACATTTGC
GAACTGGCCGCGAGGATGCTCTTCAGCGCCGTCGAGTGGGCCCGGAA
CATCCCCTTCTTCCCCGACCTGCAGATCACGGACCAGGTGGCCCTGCT
TCGCCTCACCTGGAGCGAGCTGTTTGTGTTGAATGCGGCGCAGTGCTC
CATGCCCCTCCACGTCGCCCCGCTCCTGGCCGCCGCCGGCCTGCATGC
TTCGCCCATGTCCGCCGACCGGGTGGTCGCCTTTATGGACCACATA
CGGATCTTCCAAGAGCAAGTGGAGAAGCTCAAGGCGCTGCACGTTGA
CTCAGCCGAGTACAGCTGCCTCAAGGCCATAGTCCTGTTCACCTCAGA
TGCCTGTGGTCTCTCTGATGTAGCCCATGTGGAAAGCTTGCAGGAAAA
GTCTCAGTGTGCTTTGGAAGAATACGTTAGGAGCCAGTACCCCAACCA
GCCGACGAGATTCGGAAAGCTTTTGCTTCGCCTCCCTTCCCTCCGCACC
GTCTCCTCCTCAGTCATAGAGCAATTGTTTTTCGTCCGTTTGGTAGGTA
AAACCCCCATCGAAACCCTCATCCGGGATATGTTACTGTCCGGCAGCA
GTTTTAACTGGCCGTATATGGCAATTCAATAAATAAATAAAATAAGAA
GGGGGAGTGAAACAGAGAAAGAAAAGGCAAAAGACTGGTTTGTTTGC
TTAATTTCCTTCTGTTAAGAAAGGATATAAAAGGATGTTACAAGTTTG
CTAAAAGAAGAGAGGGGAAGAATTTAATGGACTGTGAATTTCAAAAA
AAAAAAAAAAGACTGTCAAATGAACTTTTACAGAATGCATTAAAAAA
AAAAAAAAACTCCTGTGTCGGTCAGAACAACTTGCTACTTATCATTTT
TGTATAAAAAGGAAATTAGTCTTTTTCTTTTTTTGGTAAATTTTTGAAA
AATATTGCTAAAAGTGCATTTAAGGAGATTGGGAGACAATTAGCAGA
ATGGAGAAAGTAAGTCTTTTTTTTTTCCAAATTATTAATTGTCCTGTGT
CTATGTACCTCTAGCTGTTCTTTTTTGTACTTTTCTGGTTCCAAACCAGT
TTATTCTGTGGTTCTATAATAAGTTTTGATATAATCTTGGCTTCTTAAA
AACTGTGTATCATTAAAATATATGTTCTGCAAGAATTAAAACTGAGTC
CATGAAAATACCATAGGAAGACATAAAACTTTAAAAGGCAACTCAAA
GATGATGGAAACGCACTTACAAGTGGTGACCAAAATTTTTAGGTGAAG
TCGAGCACTCTAATTAGAGAACTGGAGGAACCACATATAACACTTAAC
TTCCCCTACCCTGCCCCTCCCCAAAAGAAACCATGACAAACCTAGCTT
TTAAAAAATATTTTAAGAAAGAGAATGAACTGTGGAATTTATTGGCAG
CCAAGGAATGTGTCCAAGACACATGCTGAGGTTTTGAATAAAAAGTG
AACTTTTGTAATTTGAATTGGGTCCCGCTTAGTTCTTGAATTGTTATGA
AAATCCTATATCTGTTTGTATATTTGCAAACCCTTTGTATTATAATTGT
TGATATTTTCCCTTTTTAAAAAATACCATTGAAATCAGCATGACAAAA
ATAACACTGTTGGCACTTATAGGTAACGTGATTGATTCAGTATCTTAG
AGTTTACAGTTTGTGTTTTAAAAAAACTGAAGGTTTTTTTTTTAAGTGC
AACATTTCTGTATACTGTAAAAGTTATAATAACTGAACTGTTTGGTCG
AGTCTTTGTGTGTTATATTCCAAGGAAAATTGAAAGTATTCAGAAATT
AAAATATTATTTGATATCTGAAACCTGGCTGTCCCCACTCACTGTCTTT
ACATCTAGAAGAGCCCCTGTGAGCTCTCGCTTAGCTGGCCGGGCGGGG
GGTGGTGGGGGGGGGCATTTGTTTACTCCCCTCAGTCAGTTTGTTCAA
AGGTGGACTACTGTATTTGCCTGTTTAATTTGGGTGTGTGTGTGTTGGG
GGGGGAGCTGAAGTTAATGGTTTATCTATGGTTTAGGAAGTGCCATAC
TGATATAGTAAACCACCCCCATTCACCTAATCCTCCTTTTAATTAAAAA
TGGATTTTCCAGGAAAAAAAAAAAGGCCCTTATATTTGTCACACTTAA
GTGCCTGCTTAGGGAAGGTATTGTGAAAAAGTATTAGAAATCTTGAGA
TCAGTATCTATTTTATGATCAGAAAAAAATACTCTTTTGTACATTTCTG
ACAGTTACTCAGAAGATCGTTCAAGCAAGCTAATCACAGCATTGTAAC
TAGAGGACAGTTGTTTGCAGTGAGTTTTTCCTTAAGTAGGTACGATTTT
TTAAAATATTCTGTGATTCTACTCTAGCGTGGTTGTTGAGAGAGTTTCA
AATTCAGTGATACAGGTTCTAAGACTGAAAGGTCTACTTTTAATGTAT
TATGATAACTTGCAGTTGGTTTCCCTCTCCCCTCCCCCCCTTTACCTTC
AGTCTGTGAGAGCATGACCACAGGGTCAAGGGAATCTTTTCCATTGGA
GTTATGTACATAAAAACACATCGACATTTTGACATTTCAGATTGTGTG
CTACAATCTGTACTGCTCTTGGGATCCTTTGTCCTTAGAAGCCAAATTA
AGGAAGAGAAAGCAGGACAGAGAAAAAGAAAGAAGGAAGGAGGGA
AACTTTACAGGGTGTGCTGATTTGGAAGTAGTAACTATTTCTTTTGGAG
TCTTTTTTTCATTTTTCCTCTTTCTCTTTTCCTGGTTTGGAGGAAGCTCG
GTGCTGGGAGCTTGCAATTTTGTTCTTATTCAAGGTTTCCAACCCACCC
CCCCACCGCCAGTACTTCATCATGTTGTGGTTTAATTCTAATTGGTGGG
GGGGGGGGAGGACTAGTGAGGGAGGTGAAAGAACAGGGATAATTTTG
TAAAGTGTATTAAACGTTAATATTCAGATCCAGTCAATACATGCAGAC
CAGTAAAATCTGATTTGTGCAGAGTTCTCCATCTGACTCTCACTTATTT
CTGTAGATATATACATATATAAATACAAGTATGTTCTTACGGCACAGT
ATTGCTGACCTTTAGTTCGAGGTTTTGTCGGTTGTTGTTGATTTTCTTCC
TCTTGCAAGTGCTATCCATGTGAGTGTGTGAAGTTTCTCTAATAAGTAA
AACACAGGCCCTTTTCCTTGTTTGTTTTGTGTTAGTTTATTGTAAACAG
CCATTTGTTGTAAATTATTATTGGCATTAAATTATAATTTATGATTTTC
AAAGCAAAAGACAA
SEQ ID NO: 2
Homo sapiens nuclear receptor subfamily 2,
group F, member 2 (NR2F2), transcript
variant 2, mRNA
NCBI Reference Sequence: NM_001145155.1
>gi|223555948|ref|NM_001145155.1| Homo sapiens
nuclear receptor subfamily 2, group F, member
2 (NR2F2), transcript variant 2, mRNA
ATGAGAGACAAGGATCACTCCAGACATCTCCTACCTACGGTTTGGGGT
TTTTTTTCTTAAAGGCGAGGCTTGCATTCCTCAGCAGCTATGTACAAAG
CTCCCTGAAACCTTGTCTCTCTAAAGTTAGTGTGCAGGGTTTTCCAAGG
CTGAGAGAGCCTAATACATGGGGAAGCACTTCCTTGAGGTGGAAGAT
CTCTCCCTTCACCTTTCCTCTTTTTCCCTGCAGGCTAGTGCCTACTTTTT
ATCAGTTTGCACAATCGCTTAGATAAACACCGAGGAGGAGATTCTCTT
TAATTATCAAAGACACATCTTTTCAGGGGGCCAACAAAGCATTTATTT
CACCCGCCAAACTAAAGGAGAGTTATTCCAGTTTAGGAGGAAGATGC
AAGCGGTTTGGGACCTTGAACAAGGCAAATATGGTTTTGCGGTGCAGA
GGGGCAGGATGCCGCCGACCCAGCCGACCCACGGGCAGTTCGCGCTG
ACCAACGGGGATCCCCTCAACTGCCACTCGTACCTGTCCGGATATATT
TCCCTGCTGTTGCGCGCGGAGCCCTATCCCACGTCGCGCTTCGGCAGC
CAATGCATGCAGCCCAACAACATCATGGGTATCGAGAACATTTGCGAA
CTGGCCGCGAGGATGCTCTTCAGCGCCGTCGAGTGGGCCCGGAACATC
CCCTTCTTCCCCGACCTGCAGATCACGGACCAGGTGGCCCTGCTTCGC
CTCACCTGGAGCGAGCTGTTTGTGTTGAATGCGGCGCAGTGCTCCATG
CCCCTCCACGTCGCCCCGCTCCTGGCCGCCGCCGGCCTGCATGCTTCG
CCCATGTCCGCCGACCGGGTGGTCGCCTTTATGGACCACATACGGATC
TTCCAAGAGCAAGTGGAGAAGCTCAAGGCGCTGCACGTTGACTCA
GCCGAGTACAGCTGCCTCAAGGCCATAGTCCTGTTCACCTCAGATGCC
TGTGGTCTCTCTGATGTAGCCCATGTGGAAAGCTTGCAGGAAAAGTCT
CAGTGTGCTTTGGAAGAATACGTTAGGAGCCAGTACCCCAACCAGCCG
ACGAGATTCGGAAAGCTTTTGCTTCGCCTCCCTTCCCTCCGCACCGTCT
CCTCCTCAGTCATAGAGCAATTGTTTTTCGTCCGTTTGGTAGGTAAAAC
CCCCATCGAAACCCTCATCCGGGATATGTTACTGTCCGGCAGCAGTTT
TAACTGGCCGTATATGGCAATTCAATAAATAAATAAAATAAGAAGGG
GGAGTGAAACAGAGAAAGAAAAGGCAAAAGACTGGTTTGTTTGCTTA
ATTTCCTTCTGTTAAGAAAGGATATAAAAGGATGTTACAAGTTTGCTA
AAAGAAGAGAGGGGAAGAATTTAATGGACTGTGAATTTCAAAAAAAA
AAAAAAAGACTGTCAAATGAACTTTTACAGAATGCATTAAAAAAAAA
AAAAAACTCCTGTGTCGGTCAGAACAACTTGCTACTTATCATTTTTGTA
TAAAAAGGAAATTAGTCTTTTTCTTTTTTTGGTAAATTTTTGAAAAATA
TTGCTAAAAGTGCATTTAAGGAGATTGGGAGACAATTAGCAGAATGG
AGAAAGTAAGTCTTTTTTTTTTCCAAATTATTAATTGTCCTGTGTCTAT
GTACCTCTAGCTGTTCTTTTTTGTACTTTTCTGGTTCCAAACCAGTTTAT
TCTGTGGTTCTATAATAAGTTTTGATATAATCTTGGCTTCTTAAAAACT
GTGTATCATTAAAATATATGTTCTGCAAGAATTAAAACTGAGTCCATG
AAAATACCATAGGAAGACATAAAACTTTAAAAGGCAACTCAAA
GATGATGGAAACGCACTTACAAGTGGTGACCAAAATTTTTAGGTGAAG
TCGAGCACTCTAATTAGAGAACTGGAGGAACCACATATAACACTTAAC
TTCCCCTACCCTGCCCCTCCCCAAAAGAAACCATGACAAACCTA
GCTTTTAAAAAATATTTTAAGAAAGAGAATGAACTGTGGAATTTATTG
GCAGCCAAGGAATGTGTCCAAGACACATGCTGAGGTTTTGAATAAAA
AGTGAACTTTTGTAATTTGAATTGGGTCCCGCTTAGTTCTTGAATTGTT
ATGAAAATCCTATATCTGTTTGTATATTTGCAAACCCTTTGTATTATAA
TTGTTGATATTTTCCCTTTTTAAAAAATACCATTGAAATCAGCATGACA
AAAATAACACTGTTGGCACTTATAGGTAACGTGATTGATTCAGTATCT
TAGAGTTTACAGTTTGTGTTTTAAAAAAACTGAAGGTTTTTTTTTTAAG
TGCAACATTTCTGTATACTGTAAAAGTTATAATAACTGAACTGTTTGGT
CGAGTCTTTGTGTGTTATATTCCAAGGAAAATTGAAAGTATTCAGAAA
TTAAAATATTATTTGATATCTGAAACCTGGCTGTCCCCACTCACTGTCT
TTACATCTAGAAGAGCCCCTGTGAGCTCTCGCTTAGCTGGCCGGGCGG
GGGGTGGTGGGGGGGGGCATTTGTTTACTCCCCTCAGTCAGTTTGTTC
AAAGGTGGACTACTGTATTTGCCTGTTTAATTTGGGTGTGTGTGTGTTG
GGGGGGGAGCTGAAGTTAATGGTTTATCTATGGTTTAGGAAGTGCCAT
ACTGATATAGTAAACCACCCCCATTCACCTAATCCTCCTTTTAATTAAA
AATGGATTTTCCAGGAAAAAAAAAAAGGCCCTTATATTTGTCACACTT
AAGTGCCTGCTTAGGGAAGGTATTGTGAAAAAGTATTAGAAATCTTGA
GATCAGTATCTATTTTATGATCAGAAAAAAATACTCTTTTGTACATTTC
TGACAGTTACTCAGAAGATCGTTCAAGCAAGCTAATCACAGCATTGTA
ACTAGAGGACAGTTGTTTGCAGTGAGTTTTTCCTTAAGTAGGTACGAT
TTTTTAAAATATTCTGTGATTCTACTCTAGCGTGGTTGTTGAGAGAGTT
TCAAATTCAGTGATACAGGTTCTAAGACTGAAAGGTCTACTTTTAATG
TATATATGATAACTTGCAGTTGGTTTCCCTCTCCCCTCCCCCCCTTTAC
CTTCAGTCTGTGAGAGCATGACCACAGGGTCAAGGGAATCTTTTCCAT
TGGAGTTATGTACATAAAAACACATCGACATTTTGACATTTCAGATTG
TGTGGCTACAATCTGTACTGCTCTTGGGATCCTTTGTCCTTAGAAGCCA
AATTAAGGAAGAGAAAGCAGGACAGAGAAAAAGAAAGAAGGAAGGA
GGGAAACTTTACAGGGTGTGCTGATTTGGAAGTAGTAACTATTTCTTTT
GGAGTCTTTTTTTCATTTTTCCTCTTTCTCTTTTCCTGGTTTGGAGGAAG
CTCGGTGCTGGGAGCTTGCAATTTTGTTCTTATTCAAGGTTTCCAACCC
ACCCCCCCACCGCCAGTACTTCATCATGTTGTGGTTTAATTCTAATTGG
TGGGGGGGGGGGAGGACTAGTGAGGGAGGTGAAAGAACAGGGATAA
TTTTGTAAAGTGTATTAAACGTTAATATTCAGATCCAGTCAATACATGC
AGACCAGTAAAATCTGATTTGTGCAGAGTTCTCCATCTGACTCTCACTT
ATTTCTGTAGATATATACATATATAAATACAAGTATGTTCTTACGGCAC
AGTATTGCTGACCTTTAGTTCGAGGTTTTGTCGGTTGTTGTTGATTTTCT
TCCTCTTGCAAGTGCTATCCATGTGAGTGTGTGAAGTTTCTCTAATAAG
TAAAACACAGGCCCTTTTCCTTGTTTGTTTTGTGTTAGTTTATTGTAAA
CAGCCATTTGTTGTAAATTATTATTGGCATTAAATTATAATTTATGA
TTTTCAAAGCAAAAGACAA
SEQ ID NO: 3
Homo sapiens nuclear receptor subfamily 2,
group F, member 2 (NR2F2), transcript
variant 3, mRNA
NCBI Reference Sequence: NM_001145156.1
>gi|223555950|ref|NM_001145156.1| Homo sapiens
nuclear receptor subfamily 2, group F, member
2 (NR2F2), transcript variant 3, mRNA
CTCCTTCCCTCGTCCTGGGTCCCGGGGTCCTGGGTACGTTTGGCTAGCC
TGCTCTGGCGGTGCAGAGGGGCAGGATGCCGCCGACCCAGCCGACCC
ACGGGCAGTTCGCGCTGACCAACGGGGATCCCCTCAACTGCCACTCGT
ACCTGTCCGGATATATTTCCCTGCTGTTGCGCGCGGAGCCCTATCCCAC
GTCGCGCTTCGGCAGCCAATGCATGCAGCCCAACAACATCATGGGTAT
CGAGAACATTTGCGAACTGGCCGCGAGGATGCTCTTCAGCGCCGTCGA
GTGGGCCCGGAACATCCCCTTCTTCCCCGACCTGCAGATCACGGACCA
GGTGGCCCTGCTTCGCCTCACCTGGAGCGAGCTGTTTGTGTTGAATGC
GGCGCAGTGCTCCATGCCCCTCCACGTCGCCCCGCTCCTGGCCGCCGC
CGGCCTGCATGCTTCGCCCATGTCCGCCGACCGGGTGGTCGCCTTTAT
GGACCACATACGGATCTTCCAAGAGCAAGTGGAGAAGCTCAAGGCGC
TGCACGTTGACTCAGCCGAGTACAGCTGCCTCAAGGCCATAGTCCTGT
TCACCTCAGATGCCTGTGGTCTCTCTGATGTAGCCCATGTGGAAAGCTT
GCAGGAAAAGTCTCAGTGTGCTTTGGAAGAATACGTTAGGAGCCAGT
ACCCCAACCAGCCGACGAGATTCGGAAAGCTTTTGCTTCGCCTCCCTT
CCCTCCGCACCGTCTCCTCCTCAGTCATAGAGCAATTGTTTTTCGTCCG
TTTGGTAGGTAAAACCCCCATCGAAACCCTCATCCGGGATATGTTACT
GTCCGGCAGCAGTTTTAACTGGCCGTATATGGCAATTCAATAAATAAA
TAAAATAAGAAGGGGGAGTGAAACAGAGAAAGAAAAGGCAAAAGA
CTGGTTTGTTTGCTTAATTTCCTTCTGTTAAGAAAGGATATAAAAGGAT
GTTACAAGTTTGCTAAAAGAAGAGAGGGGAAGAATTTAATGGACTGT
GAATTTCAAAAAAAAAAAAAAAGACTGTCAAATGAACTTTTACA
GAATGCATTAAAAAAAAAAAAAAACTCCTGTGTCGGTCAGAACAACT
TGCTACTTATCATTTTTGTATAAAAAGGAAATTAGTCTTTTTCTTTTTTT
GGTAAATTTTTGAAAAATATTGCTAAAAGTGCATTTAAGGAGATTGGG
AGACAATTAGCAGAATGGAGAAAGTAAGTCTTTTTTTTTTCCAAATTA
TTAATTGTCCTGTGTCTATGTACCTCTAGCTGTTCTTTTTTGTACTTTTC
TGGTTCCAAACCAGTTTATTCTGTGGTTCTATAATAAGTTTTGATATAA
TCTTGGCTTCTTAAAAACTGTGTATCATTAAAATATATGTTCTGCAAGA
ATTAAAACTGAGTCCATGAAAATACCATAGGAAGACATAAAACTTTA
AAAGGCAACTCAAAGATGATGGAAACGCACTTACAAGTGGTGACCAA
AATTTTTAGGTGAAGTCGAGCACTCTAATTAGAGAACTGGAGGAACCA
CATATAACACTTAACTTCCCCTACCCTGCCCCTCCCCAAAAGAAACCA
TGACAAACCTAGCTTTTAAAAAATATTTTAAGAAAGAGAATGAACTGT
GGAATTTATTGGCAGCCAAGGAATGTGTCCAAGACACATGCTGAGGTT
TTGAATAAAAAGTGAACTTTTGTAATTTGAATTGGGTCCCGCTTAGTTC
TTGAATTGTTATGAAAATCCTATATCTGTTTGTATATTTGCAAACCCTT
TGTATTATAATTGTTGATATTTTCCCTTTTTAAAAAATACCATTGAAAT
CAGCATGACAAAAATAACACTGTTGGCACTTATAGGTAACGTGATTGA
TTCAGTATCTTAGAGTTTACAGTTTGTGTTTTAAAAAAACTGAAGGTTT
TTTTTTTAAGTGCAACATTTCTGTATACTGTAAAAGTTATAATAACTGA
ACTGTTTGGTCGAGTCTTTGTGTGTTATATTCCAAGGAAAATTGAAAGT
ATTCAGAAATTAAAATATTATTTGATATCTGAAACCTGGCTGTCCCCA
CTCACTGTCTTTACATCTAGAAGAGCCCCTGTGAGCTCTCGCTTAGCTG
GCCGGGCGGGGGGTGGTGGGGGGGGGCATTTGTTTACTCCCCTCAGTC
AGTTTGTTCAAAGGTGGACTACTGTATTTGCCTGTTTAATTTGGGTGTG
TGTGTGTTGGGGGGGGAGCTGAAGTTAATGGTTTATCTATGGTTTAGG
AAGTGCCATACTGATATAGTAAACCACCCCCATTCACCTAATCCTCCT
TTTAATTAAAAATGGATTTTCCAGGAAAAAAAAAAAGGCCCTTATATT
TGTCACACTTAAGTGCCTGCTTAGGGAAGGTATTGTGAAAAAGTATTA
GAAATCTTGAGATCAGTATCTATTTTATGATCAGAAAAAAATACT
CTTTTGTACATTTCTGACAGTTACTCAGAAGATCGTTCAAGCAAGCTA
ATCACAGCATTGTAACTAGAGGACAGTTGTTTGCAGTGAGTTTTTCCTT
AAGTAGGTACGATTTTTTAAAATATTCTGTGATTCTACTCTAGCGTGGT
TGTTGAGAGAGTTTCAAATTCAGTGATACAGGTTCTAAGACTGAAAGG
TCTACTTTTAATGTATATATGATAACTTGCAGTTGGTTTCCCTCTCCCCT
CCCCCCCTTTACCTTCAGTCTGTGAGAGCATGACCACAGGGTCAAGGG
AATCTTTTCCATTGGAGTTATGTACATAAAAACACATCGACATTTTGAC
ATTTCAGATTGTGTGGCTACAATCTGTACTGCTCTTGGGATCCTTTGTC
CTTAGAAGCCAAATTAAGGAAGAGAAAGCAGGACAGAGAAAAAGAA
AGAAGGAAGGAGGGAAACTTTACAGGGTGTGCTGATTTGGAAGTAGT
AACTATTTCTTTTGGAGTCTTTTTTTCATTTTTCCTCTTTCTCTTTTCCT
GGTTTGGAGGAAGCTCGGTGCTGGGAGCTTGCAATTTTGTTCTTATTCAA
GGTTTCCAACCCACCCCCCCACCGCCAGTACTTCATCATGTTGTGGT
TTAATTCTAATTGGTGGGGGGGGGGGAGGACTAGTGAGGGAGGTGAA
AGAACAGGGATAATTTTGTAAAGTGTATTAAACGTTAATATTCAGATC
CAGTCAATACATGCAGACCAGTAAAATCTGATTTGTGCAGAGTTCTCC
ATCTGACTCTCACTTATTTCTGTAGATATATACATATATAAATACAAGT
ATGTTCTTACGGCACAGTATTGCTGACCTTTAGTTCGAGGTTTTGTCGG
TTGTTGTTGATTTTCTTCCTCTTGCAAGTGCTATCCATGTGAGTGTGTG
AAGTTTCTCTAATAAGTAAAACACAGGCCCTTTTCCTTGTTTGTTTTGT
GTTAGTTTATTGTAAACAGCCATTTGTTGTAAATTATTATTGGCATTAA
ATTATAATTTATGATTTTCAAAGCAAAAGACA
A
SEQ ID NO: 4
Homo sapiens nuclear receptor subfamily 2,
group F, member 2 (NR2F2), transcript
variant 4, mRNA
NCBI Reference Sequence: NM_001145157.1
>gi|223555952|ref|NM_001145157.1| Homo sapiens
nuclear receptor subfamily 2, group F, member
2 (NR2F2), transcript variant 4, mRNA
GGTCCGGAGTCAGATAACAGCCTGGGCCCGAGCCTCGCCGGCTTTCCC
CGGCCCTTACAGGCCCTGCCCAGGCTCCGCTAGTGCCGGCCGCCTGCT
CCCTGCCTCTCCCGGCTTCCTCTCTCTTTAGCCGGCCTCTCTCTCTCCGC
CCTCTCCCTCCGTCTCTTTCTCCGAGCACACTGATTAGACAGACGCCAG
ACCTCCGCTCTCTGCTTGTCTCTCACTGGGGGGGTTCCCCGCCGGGCTG
GGGCTGGGGCTTCGGGGTTTGTGGGAGAGTCGTTCCGGAGTGGCCACA
GGCCGTCTGGGGTGGACCCTCGTGCCTTTTGCAAAAGCGCCTCACCCT
CCCCCCAGACTCGCCCCTCCCGCTCCCTCTCCTCCAATCAATAAGAAA
TATCAGCTGTTTAGCAGTAAAGAAGAAAGATGCCCTCAGAATGCTACA
TCCCGCCCACAGCGCCGGGGACCCCGAGGCAAGGTGGCCAATTCTGG
GTCCTCGGCGGACCAGCCCCGAGCGGGCCTCGGAGCGGTGCAGAGGG
GCAGGATGCCGCCGACCCAGCCGACCCACGGGCAGTTCGCGCTGACC
AACGGGGATCCCCTCAACTGCCACTCGTACCTGTCCGGATATATTTCC
CTGCTGTTGCGCGCGGAGCCCTATCCCACGTCGCGCTTCGGCAGCCAA
TGCATGCAGCCCAACAACATCATGGGTATCGAGAACATTTGCGAACTG
GCCGCGAGGATGCTCTTCAGCGCCGTCGAGTGGGCCCGGAACATCCCC
TTCTTCCCCGACCTGCAGATCACGGACCAGGTGGCCCTGCTTCGCCTC
ACCTGGAGCGAGCTGTTTGTGTTGAATGCGGCGCAGTGCTCCATGCCC
CTCCACGTCGCCCCGCTCCTGGCCGCCGCCGGCCTGCATGCTTCGCCC
ATGTCCGCCGACCGGGTGGTCGCCTTTATGGACCACATACGGATCTTC
CAAGAGCAAGTGGAGAAGCTCAAGGCGCTGCACGTTGACTCAGCCGA
GTACAGCTGCCTCAAGGCCATAGTCCTGTTCACCTCAGATGCCTGTGG
TCTCTCTGATGTAGCCCATGTGGAAAGCTTGCAGGAAAAGTCTCAGTG
TGCTTTGGAAGAATACGTTAGGAGCCAGTACCCCAACCAGCCGACGA
GATTCGGAAAGCTTTTGCTTCGCCTCCCTTCCCTCCGCACCGTCTCCTC
CTCAGTCATAGAGCAATTGTTTTTCGTCCGTTTGGTAGGTAAAACCCCC
ATCGAAACCCTCATCCGGGATATGTTACTGTCCGGCAGCAGTTTTAAC
TGGCCGTATATGGCAATTCAATAAATAAATAAAATAAGAAGGGGGAG
TGAAACAGAGAAAGAAAAGGCAAAAGACTGGTTTGTTTGCTTAATTTC
CTTCTGTTAAGAAAGGATATAAAAGGATGTTACAAGTTTGCTAAAAGA
AGAGAGGGGAAGAATTTAATGGACTGTGAATTTCAAAAAAAAAAAAA
AAGACTGTCAAATGAACTTTTACAGAATGCATTAAAAAAAAAAAAAA
ACTCCTGTGTCGGTCAGAACAACTTGCTACTTATCATTTTTGTATAAAA
AGGAAATTAGTCTTTTTCTTTTTTTGGTAAATTTTTGAAAAATATTGCT
AAAAGTGCATTTAAGGAGATTGGGAGACAATTAGCAGAATGGAGAAA
GTAAGTCTTTTTTTTTTCCAAATTATTAATTGTCCTGTGTCTATGTACCT
CTAGCTGTTCTTTTTTGTACTTTTCTGGTTCCAAACCAGTTTATTCTGTG
GTTCTATAATAAGTTTTGATATAATCTTGGCTTCTTAAAAACTGTGTAT
CATTAAAATATATGTTCTGCAAGAATTAAAACTGAGTCCATGAAAATA
CCATAGGAAGACATAAAACTTTAAAAGGCAACTCAAAGATGATGGAA
ACGCACTTACAAGTGGTGACCAAAATTTTTAGGTGAAGTCGAGCACTC
TAATTAGAGAACTGGAGGAACCACATATAACACTTAACTTCCCCTACC
CTGCCCCTCCCCAAAAGAAACCATGACAAACCTAGCTTTTAAAAAATA
TTTTAAGAAAGAGAATGAACTGTGGAATTTATTGGCAGCCAAGGAATG
TGTCCAAGACACATGCTGAGGTTTTGAATAAAAAGTGAACTTTTGTAA
TTTGAATTGGGTCCCGCTTAGTTCTTGAATTGTTATGAAAATCCTATAT
CTGTTTGTATATTTGCAAACCCTTTGTATTATAATTGTTGATATTTTCCC
TTTTTAAAAAATACCATTGAAATCAGCATGACAAAAATAACACTGTTG
GCACTTATAGGTAACGTGATTGATTCAGTATCTTAGAGTTTACAGTTTG
TGTTTTAAAAAAACTGAAGGTTTTTTTTTTAAGTGCAACATTTCTGTAT
ACTGTAAAAGTTATAATAACTGAACTGTTTGGTCGAGTCTTTGTGTGTT
ATATTCCAAGGAAAATTGAAAGTATTCAGAAATTAAAATATTATTTGA
TATCTGAAACCTGGCTGTCCCCACTCACTGTCTTTACATCTAGAAGAGC
CCCTGTGAGCTCTCGCTTAGCTGGCCGGGCGGGGGGTGGTGGGGGGG
GGCATTTGTTTACTCCCCTCAGTCAGTTTGTTCAAAGGTGGACTACTGT
ATTTGCCTGTTTAATTTGGGTGTGTGTGTGTTGGGGGGGGAGCTGAAG
TTAATGGTTTATCTATGGTTTAGGAAGTGCCATACTGATATAGTAAAC
CACCCCCATTCACCTAATCCTCCTTTTAATTAAAAATGGATTTTCCAGG
AAAAAAAAAAAGGCCCTTATATTTGTCACACTTAAGTGCCTGCTTAGG
GAAGGTATTGTGAAAAAGTATTAGAAATCTTGAGATCAGTATCTATTT
TATGATCAGAAAAAAATACTCTTTTGTACATTTCTGACAGTTACTCAG
AAGATCGTTCAAGCAAGCTAATCACAGCATTGTAACTAGAGGACAGTT
GTTTGCAGTGAGTTTTTCCTTAAGTAGGTACGATTTTTTAAAATATTCT
GTGATTCTACTCTAGCGTGGTTGTTGAGAGAGTTTCAAATTCAGTGAT
ACAGGTTCTAAGACTGAAAGGTCTACTTTTAATGTATATATGATAACT
TGCAGTTGGTTTCCCTCTCCCCTCCCCCCCTTTACCTTCAGTCTGTGAG
AGCATGACCACAGGGTCAAGGGAATCTTTTCCATTGGAGTTATGTACA
TAAAAACACATCGACATTTTGACATTTCAGATTGTGTGGCTACAATCT
GTACTGCTCTTGGGATCCTTTGTCCTTAGAAGCCAAATTAAGGAAGAG
AAAGCAGGACAGAGAAAAAGAAAGAAGGAAGGAGGGAAACTTTACA
GGGTGTGCTGATTTGGAAGTAGTAACTATTTCTTTTGGAGTCTTTTTTT
CATTTTTCCTCTTTCTCTTTTCCTGGTTTGGAGGAAGCTCGGTGCTGGG
AGCTTGCAATTTTGTTCTTATTCAAGGTTTCCAACCCACCCCCCCACCG
CCAGTACTTCATCATGTTGTGGTTTAATTCTAATTGGTGGGGGGGGGG
GAGGACTAGTGAGGGAGGTGAAAGAACAGGGATAATTTTGTAAAGTG
TATTAAACGTTAATATTCAGATCCAGTCAATACATGCAGACCAGTAAA
ATCTGATTTGTGCAGAGTTCTCCATCTGACTCTCACTTATTTCTGTAGA
TATATACATATATAAATACAAGTATGTTCTTACGGCACAGTATTGCTG
ACCTTTAGTTCGAGGTTTTGTCGGTTGTTGTTGATTTTCTTCCTCTTGCA
AGTGCTATCCATGTGAGTGTGTGAAGTTTCTCTAATAAGTAAAACACA
GGCCCTTTTCCTTGTTTGTTTTGTGTTAGTTTATTGTAAACAGCCATTTG
TTGTAAATTATTATTGGCATTAAATTATAATTTATGATTTTCAAAGCAA
AAGACAA
SEQ ID NO: 5
Mus musculus nuclear receptor subfamily 2,
group F, member 2 (Nr2f2), transcript
variant 1, mRNA
NCBI Reference Sequence: NM_009697.3
>gi|112421175|ref|NM_009697.3| Mus musculus
nuclear receptor subfamily 2, group F,
member 2 (Nr2f2), transcript variant 1,
mRNA
CGAGGGAAACAAACAAAACAAACACACCGGGCCAGACAAGCAATCG
ACAAAACTTTGCAAAAGCAAAAACAAAAAAACAAAAAAGGAAAAAC
TAACCAACCTCAAATCAACTAGCCCTGAGCCACCCGGGGCGCCCTCCC
GCGCCCTCTCGCACCCTCGCACACACAAAAGGCGGCGCGCCGGAGCC
CGAGACCCGGGAGCCGCCGCCACCCCGCCGCCGCCCGCAGCCAGGGG
AGCAGAAGTCCGGACGCGGCCCCCATAGATATGGCAATGGTAGTCA
GCACGTGGCGCGACCCCCAGGACGAGGTGCCCGGCTCTCAGGGCAGC
CAGGCCTCGCAGGCGCCGCCCGTGCCGGGCCCGCCGCCTGGCGCCCCG
CACACGCCACAGACGCCGGGCCAAGGGGGCCCGGCCAGCACGCCG
GCCCAGACAGCGGCTGGCGGCCAGGGCGGCCCTGGCGGCCCGGGCAG
CGACAAGCAGCAGCAGCAGCAGCACATCGAGTGCGTGGTGTGCGGGG
ACAAGTCGAGCGGCAAGCACTACGGCCAGTTCACGTGCGAGGGCTG
CAAGAGCTTCTTCAAGCGCAGCGTGCGGAGGAACCTGAGCTACACGT
GCCGCGCCAACCGGAACTGTCCCATCGACCAGCACCACCGCAACCAG
TGCCAGTACTGCCGCCTCAAAAAGTGCCTCAAAGTGGGCATGAGACG
GGAAGCTGTACAGAGAGGCAGGATGCCTCCTACCCAGCCTACCCACG
GGCAGTTTGCCCTGACCAACGGGGACCCCCTCAACTGCCACTCGTACC
TGTCCGGATATATTTCCCTGCTGCTGCGCGCGGAGCCCTACCCCACGT
CGCGCTTCGGCAGTCAGTGCATGCAGCCTAACAACATCATGGGCATCG
AGAACATTTGCGAACTGGCCGCACGGATGCTCTTCAGCGCCGTTGAGT
GGGCCCGGAACATCCCCTTCTTCCCTGACCTGCAGATCACGGACCAGG
TGGCCCTCCTTCGCCTCACCTGGAGCGAGCTGTTCGTGTTGAATGCGG
CCCAGTGCTCCATGCCCCTCCATGTCGCCCCGCTCCTTGCCGCTGCTGG
CCTGCACGCTTCACCCATGTCAGCCGACCGGGTGGTCGCTTTTATGGA
CCACATACGGATCTTCCAAGAGCAAGTGGAGAAGCTCAAGGCACTGC
ACGTCGACTCCGCCGAGTATAGCTGCCTCAAGGCCATAGTCCTGTTCA
CCTCAGATGCCTGTGGTCTGTCTGATGTAGCCCATGTGGAAAGCTTGC
AGGAAAAGTCCCAGTGTGCTTTGGAAGAGTACGTTAGGAGCCAGTAC
CCCAACCAGCCAACACGGTTCGGAAAGCTCTTGCTTCGTCTCCCTTCC
CTCCGCACGGTCTCCTCCTCAGTCATAGAGCAATTGTTTTTCGTCCGTT
TGGTAGGTAAAACCCCCATCGAAACCCTCATCCGGGATATGTTACTGT
CCGGCAGCAGTTTTAACTGGCCATATATGGCAATTCAATAAATAAATC
AATCAAAATAAGGGGGAGTGAAACAGAGAAAGAAAAGGCAAAAGAC
TGGTTTTGTTTGCTTAATTTCCTTCTGTTAAGAAAGGATGTTACAAGTT
TGCTAAAAAGAAGAGAGGGGAAGAATTTAATGGACTGTGAATTTCAA
AAAGGAGAGAGAGAAAGAGAGAGACTGCCAAATGAACTTTTACAGAA
TGCATTAAAAAAAAAGAAAGAAAACAACTCCTGTGTTGGGCAGAACA
ACCTGCTACTTATCATTTTTGTATAAAAAGGAAATTAGTCTTTTTTTCT
TTTTGGTAAATTTTTGAAAAATATTGCTAAAAGTGCATTTAAGGAGAT
TGGGAGAAAATTAGCAGAATGGACAAAGTAAGTCATTTTTTTCCAAAT
TATTAATTGTCCTGTGTCTATGTACCTCTAGTTGTTCTTTTTTTTTTTTT
TTAACTTTTCTGGTTCCAAACCAGTTTATTCTGTGGTTCAATAATAAGTT
TTGATATAATCTTGGCTTCTTAAAAACTGTGTATCATTAAAATATATGT
TCTGCAAGAATTAAAACTGAGTCCATGAAAATAGCATAGGAAAACAT
AAAACTTTAAAAGGCAACTCAGAGATGGTGGAAATGCACTTACAAGT
GGTGGCCAAATTGTTTTTTTTTTTTTTTTTTTAAGGTAAAGTTGAGCACT
CTAATTAGCAAGCTGGGGGAATCACATCAACACTTAGCTTCCCCACCC
CCACCCCATACCATGACAAACCTAGCTTTTTAAAAAAAATATTTTAAG
AAACAGAAGGAACTGTGGAATTTATTGGCAGCCAAGGAATGTGTCCA
AGACACAAGCTGAGGTTTTTGAATAAAAAGTGAACTTTTGTAATTTGA
ATTGGGTCCCCCCCCCTTAGTTCTTGAATTGTTATGAATCCTATATCTG
TTTGTATATTTGCAAGCCCTTTGTATTATAATTGTTGATATTTCCCCTTT
TTAAAAAATACCATTGAAATCAGCATGACAAAATAACACTGTTGGCAC
TTATAGGTAACGTGATTGATTCAGTATCTTAGAGTTTACAGTTTGTGTT
TTTAAAAAACTGAAGGTTTTTTTTTTAAGTGCAACATTTCTGTATACTG
TAAAAGTTATAATAACTGAACTGTTTGGTCGAGTCTTTGTGTGTTATAT
TCCAAGGAAATTGAAAGTATTCAGAAATTAAAATATTATTTGATATCT
GAAATCTGCTTGGCTGTCCCCACTCACTGTCTTTCCACGGAGCTGAGC
CCCTGTGAGTTCTCGCTGAGCCAGCGGGGGCCCCATTTGTTTACTCCCT
CAATCAGTTTGTTCAAAGGTAGACTAGTGTATTTGCCTGTTTAATTTGG
GTGTGGTGTGGGGGGGGAGCTGAAGTTAATGGTTTAGCTATGGTTTAG
GAAGTGCCACACTGATATAGTAAGCCACCCCCATTCACCTAATCCTAC
TTTTAATTAAAAATGGATTTTCCAGGAAAAAAATAAGGCCCTTATATT
TGTCACACTTAAGTGCCTGCTTAGGGAAGGTATTGTGAAAAGTATTAG
AAATTTTGAGATCAGTATCTGTTTTATGATCAGAAAAAAAATGCTCTTT
TGTACATTTGTGACAGTTATGCAGAGGACTGTCCAAGCAAGCTAATCA
CAGAACTGTAAATAGAGGGCAGTTGTTTGCAATGAGTTTTTCCTTAAG
TAAGTGTAATTTTTCTTTTTCTTTTTTTCTTTTTTTTTTAAAAATATCCT
GAGGTTCTCATTTAGCGTGGCTGTTGAGAGGATTTTGAATACAGTGATG
TAGCTGCTAGCGACGAAGGGTCTGTTTTTCTTGTATATACATGATAACT
TGCAGTTGCCCTGCCTTTCCCCTCCCCCTCCCTCTTCAGTCTGTTGAGA
GCATGGCCACAGGTCAAGGGAATCTTTACCATTGGAGTTATGTACATA
AAAAAAAAAAACCATGAACATTTGGACATTTCAGATTATATAGAAAC
AATCTGTACTGCTCTGGGATCCTTTGGTCTTAGAAACCATTTTTGGGGG
GGTGGAGAGAGAGAGAGGGAGAGGAGAGAGAGAGAGAGAGAGAGA
GAGAGAGAGAGAGAGAGAGAGAGAGAGAGAGAGAGAGAGAATAAA
GAAAACTTTACAGGGTGTGCTGATTTGGGAAGTCAACTATTTGGTTCT
GTCCCTTATTCTCTTTTCCTGGTTTGGGAAGAGCTCACTGCTGGGAACT
TGCAATTTGTTCTTATTTAGACTTTCCAAGCTGCCCTCCCTGACAATAC
TTTTACCATGTTGTGGTTTAATCTTAAAACGGGGGAGGGGGCTGGTGA
CAGAGGTGAAAGAAAGGAGATCAGTTTGCCAAGTGCATTCAACTTTG
ATGCTCAGTTCTGGTTCATACATGCAGACCTGAAAACTCTGCCTGATTT
AGGCAGAGATCTTTATCTGACCCTCAGCTTCCCTCTGTAGATATATAG
ATATATAAATATAAATATGAATATAAGTATGTTTTACAGCACAGCATC
TGACCTGTAGATGGAGGTTTTGTTGGTTGTTTATTTTCCCCTCTTGCAA
GTGCTACCCATGTGAGTGTGTGAAGTTTCTCTACTAAGTAAAACACAG
GCCCTTTTCCTTGTTTGCTTTGTGTTAGCTTATTGTAAACAGCCATTTGT
TGTAAATTATTATTGGCATTAAATTATAATTTATGATTTTCAAAGC
SEQ ID NO: 6
PREDICTED: Mus musculus nuclear receptor
subfamily 2, group F, member 2 (Nr2f2),
transcript variant X1, mRNA
NCBI Reference Sequence: XM_006540577.1
>gi|568947224|ref|XM_006540577.1| PREDICTED:
Mus musculus nuclear receptor subfamily 2,
group F, member 2 (Nr2f2), transcript
variant X1, mRNA
TTATGCACTTCGCCGTATTAACGCTGCCGCCTGGGCAGAGCTCATGTG
ACCCCTCCGTGGATTAACATTCTGCTTTAAAAAAATACCTCTTGTTTTC
TTTTTTTTTCCTTTCACTTTTGAAACCCTGAGAGCACTGTAGGGAGTAG
GAAAGTGTGGGCAAGGGGCCTTTGGCCGCTGCTTTCCCTCTGCGCCAG
TTGGGTCTTTGTGATATAAAATTATCCCAGAGCCGGGAACAGTGCTCT
ACCAATGGCGCGCTCCGCGCCTGGGCGCGGGCTCCGGGTTGGAGCGA
GCCAATGCCGGGGTTTCTTTGTGTTTCTGCGAGAGCGACTCTCCCGGTC
CTGAGTCAGATAACAGCGGGTGCCTGAGCCTCGCCGGCTTTCTCCGGT
CGTCACCGGCCTTGTCTGGGCTCCGCAAGCGCCCCACGTCTGCTCCCA
GCCTCTCTCCCGCTTCCTCTCCTCTCTGACGGCCTCACTCTTTCTCTCTC
CGCCCTTTTCTCCCTTGTCTCTCCTCCTCCGAGCTGAGCTCAGGGATCA
GGCAAAGACGCCAGACCAAGACTCTGTCTCTCGCCGGGGTTTCCTTCC
TGGGCTGGGGTTGAGGTTACAAGGTTTGGGGACAGTCGTTCGGAGGTG
GCCACAGGCCATCTGGGGTAAACCTTAATGTCTTGTGCAAAAGCGTCT
CACCCTCCCCCTACATTCCCGTCTCGTTCCTTCTCCAATCAATAAGAAA
TATCAGCTATTTAGCAGTTTTTAAAAAGAAAGAAATGAAATGAAACGA
AAGGTGCCCTAAGGATATGCTGCACCTCGCTTACAGCTCCAGGGACCC
CATTCAAAGTGACCAATTCTGGGTCCTCGGCGGACCAAGCCTAGATGG
GCCTCACAGCTGTACAGAGAGGCAGGATGCCTCCTACCCAGCCTACCC
ACGGGCAGTTTGCCCTGACCAACGGGGACCCCCTCAACTGCCACTCGT
ACCTGTCCGGATATATTTCCCTGCTGCTGCGCGCGGAGCCCTACCCCA
CGTCGCGCTTCGGCAGTCAGTGCATGCAGCCTAACAACATCATGGGCA
TCGAGAACATTTGCGAACTGGCCGCACGGATGCTCTTCAGCGCCGTTG
AGTGGGCCCGGAACATCCCCTTCTTCCCTGACCTGCAGATCACGGACC
AGGTGGCCCTCCTTCGCCTCACCTGGAGCGAGCTGTTCGTGTTGAATG
CGGCCCAGTGCTCCATGCCCCTCCATGTCGCCCCGCTCCTTGCCGCTGC
TGGCCTGCACGCTTCACCCATGTCAGCCGACCGGGTGGTCGCTTTTAT
GGACCACATACGGATCTTCCAAGAGCAAGTGGAGAAGCTCAAGGCAC
TGCACGTCGACTCCGCCGAGTATAGCTGCCTCAAGGCCATAGTCCTGT
TCACCTCAGATGCCTGTGGTCTGTCTGATGTAGCCCATGTGGAAAGCT
TGCAGGAAAAGTCCCAGTGTGCTTTGGAAGAGTACGTTAGGAGCCAGT
ACCCCAACCAGCCAACACGGTTCGGAAAGCTCTTGCTTCGTCTCCCTT
CCCTCCGCACGGTCTCCTCCTCAGTCATAGAGCAATTGTTTTTCGTCCG
TTTGGTAGGTAAAACCCCCATCGAAACCCTCATCCGGGATATGTTACT
GTCCGGCAGCAGTTTTAACTGGCCATATATGGCAATTCAATAAATAAA
TCAATCAAAATAAGGGGGAGTGAAACAGAGAAAGAAAAGGCAAAAG
ACTGGTTTTGTTTGCTTAATTTCCTTCTGTTAAGAAAGGATGTTACAAG
TTTGCTAAAAAGAAGAGAGGGGAAGAATTTAATGGACTGTGAATTTC
AAAAAGGAGAGAGAGAAAGAGAGAGACTGCCAAATGAACTTTTACAG
AATGCATTAAAAAAAAAGAAAGAAAACAACTCCTGTGTTGGGCAGAA
CAACCTGCTACTTATCATTTTTGTATAAAAAGGAAATTAGTCTTTTTTT
CTTTTTGGTAAATTTTTGAAAAATATTGCTAAAAGTGCATTTAAGGAG
ATTGGGAGAAAATTAGCAGAATGGACAAAGTAAGTCATTTTTTTCCAA
ATTATTAATTGTCCTGTGTCTATGTACCTCTAGTTGTTCTTTTTTTTTTT
TTTTAACTTTTCTGGTTCCAAACCAGTTTATTCTGTGGTTCAATAATAAG
TTTTGATATAATCTTGGCTTCTTAAAAACTGTGTATCATTAAAATATAT
GTTCTGCAAGAATTAAAACTGAGTCCATGAAAATAGCATAGGAAAAC
ATAAAACTTTAAAAGGCAACTCAGAGATGGTGGAAATGCACTTACAA
GTGGTGGCCAAATTGTTTTTTTTTTTTTTTTTTTAAGGTAAAGTTGAGC
ACTCTAATTAGCAAGCTGGGGGAATCACATCAACACTTAGCTTCCCCA
CCCCCACCCCATACCATGACAAACCTAGCTTTTTAAAAAAAATATTTT
AAGAAACAGAAGGAACTGTGGAATTTATTGGCAGCCAAGGAATGTGT
CCAAGACACAAGCTGAGGTTTTTGAATAAAAAGTGAACTTTTGTAATT
TGAATTGGGTCCCCCCCCCTTAGTTCTTGAATTGTTATGAATCCTATAT
CTGTTTGTATATTTGCAAGCCCTTTGTATTATAATTGTTGATATTTCCCC
TTTTTAAAAAATACCATTGAAATCAGCATGACAAAATAACACTGTTGG
CACTTATAGGTAACGTGATTGATTCAGTATCTTAGAGTTTACAGTTTGT
GTTTTTAAAAAACTGAAGGTTTTTTTTTTAAGTGCAACATTTCTGTATA
CTGTAAAAGTTATAATAACTGAACTGTTTGGTCGAGTCTTTGTGTGTTA
TATTCCAAGGAAATTGAAAGTATTCAGAAATTAAAATATTATTTGATA
TCTGAAATCTGCTTGGCTGTCCCCACTCACTGTCTTTCCACGGAGCTGA
GCCCCTGTGAGTTCTCGCTGAGCCAGCGGGGGCCCCATTTGTTTACTC
CCTCAATCAGTTTGTTCAAAGGTAGACTAGTGTATTTGCCTGTTTAATT
TGGGTGTGGTGTGGGGGGGGAGCTGAAGTTAATGGTTTAGCTATGGTT
TAGGAAGTGCCACACTGATATAGTAAGCCACCCCCATTCACCTAATCC
TACTTTTAATTAAAAATGGATTTTCCAGGAAAAAAATAAGGCCCTTAT
ATTTGTCACACTTAAGTGCCTGCTTAGGGAAGGTATTGTGAAAAGTAT
TAGAAATTTTGAGATCAGTATCTGTTTTATGATCAGAAAAAAAATGCT
CTTTTGTACATTTGTGACAGTTATGCAGAGGACTGTCCAAGCAAGCTA
ATCACAGAACTGTAAATAGAGGGCAGTTGTTTGCAATGAGTTTTTCCT
TAAGTAAGTGTAATTTTTCTTTTTCTTTTTTTCTTTTTTTTTTAAAAATA
TCCTGAGGTTCTCATTTAGCGTGGCTGTTGAGAGGATTTTGAATACAGT
GATGTAGCTGCTAGCGACGAAGGGTCTGTTTTTCTTGTATATACATGAT
AACTTGCAGTTGCCCTGCCTTTCCCCTCCCCCTCCCTCTTCAGTCTGTT
GAGAGCATGGCCACAGGTCAAGGGAATCTTTACCATTGGAGTTATGTA
CATAAAAAAAAAAAACCATGAACATTTGGACATTTCAGATTATATAGA
AACAATCTGTACTGCTCTGGGATCCTTTGGTCTTAGAAACCATTTTTGG
GGGGGTGGAGAGAGAGAGAGGGAGAGGAGAGAGAGAGAGAGAGAG
AGAGAGAGAGAGAGAGAGAGAGAGAGAGAGAGAGAGAGAGAGAAT
AAAGAAAACTTTACAGGGTGTGCTGATTTGGGAAGTCAACTATTTGGT
TCTGTCCCTTATTCTCTTTTCCTGGTTTGGGAAGAGCTCACTGCTGGGA
ACTTGCAATTTGTTCTTATTTAGACTTTCCAAGCTGCCCTCCCTGACAA
TACTTTTACCATGTTGTGGTTTAATCTTAAAACGGGGGAGGGGGCTGG
TGACAGAGGTGAAAGAAAGGAGATCAGTTTGCCAAGTGCATTCAACT
TTGATGCTCAGTTCTGGTTCATACATGCAGACCTGAAAACTCTGCCTG
ATTTAGGCAGAGATCTTTATCTGACCCTCAGCTTCCCTCTGTAGATATA
TAGATATATAAATATAAATATGAATATAAGTATGTTTTACAGCACAGC
ATCTGACCTGTAGATGGAGGTTTTGTTGGTTGTTTATTTTCCCCTCTTG
CAAGTGCTACCCATGTGAGTGTGTGAAGTTTCTCTACTAAGTAAAACA
CAGGCCCTTTTCCTTGTTTGCTTTGTGTTAGCTTATTGTAAACAGCCAT
TTGTTGTAAATTATTATTGGCATTAAATTATAATTTATGATTTTCAAAG
CAAAA
SEQ ID NO: 7
Mus musculus nuclear receptor subfamily 2,
group F, member 2 (Nr2f2), transcript
variant 2, mRNA
NCBI Reference Sequence: NM_183261.3
>gi|112421173|ref|NM_183261.3| Mus musculus
nuclear receptor subfamily 2, group F,
member 2 (Nr2f2), transcript variant 2, mRNA
AAATGGTGGAATTTGGCTGTGCCTCGGGGTTGTCCTGCTTTGCAATATT
GCCTATAGTTGTTTTCGGTTTTCTGCTAAGACTGAGCCGGGTTGCTCCA
GCCTCCGACTAAACTCATTAAGTTGGGAGATTTTTTTTTTTTTTTCAATT
GGAAGGGTGTTTTTAAAGTCTCCTCTTTCCAGCCCCAAACAAGGTGTA
ACAACGCACTCTTCCTTCTAAGGCATCAGATGAGAGACAAGGATCACT
CCAGACAGCTCCTACCTACGGTTTGGGGTTTTTTTTTCTTAAAGGCGAG
GCTTGCATTCCTCAGCAGCTATGTACAAAGCTCCCTGAAGCTGTCTCTC
TCTCTCTAAAGTTAGTGTGCAGGCTTTTCCAACGGCTGAGAGCGCCTG
GTACACAGGGAAGCAGTTCCTTGAGGTGGAAGATCTCTTCTTTCACCT
TTCTTTTTCCCTGCAGACTAATGCCTACTTTTTTATCAGTTTGCACAATC
GCTTAGATAAACACCGAGGAGGAGAGTCTCTTTAATTATCAAAGACAC
ATCTTTTCAGGGGGCCAACAAAGCATTTATTTCACCCGCCAAACTAAA
GGAGAGTTATTCCAGTTTAGAAGGAAGATGCAAGCGGTTTGGGACCTT
GAACAAGGCAAATATGGTTTTGCTGTACAGAGAGGCAGGATGCCTCCT
ACCCAGCCTACCCACGGGCAGTTTGCCCTGACCAACGGGGACCCCCTC
AACTGCCACTCGTACCTGTCCGGATATATTTCCCTGCTGCTGCGCGCG
GAGCCCTACCCCACGTCGCGCTTCGGCAGTCAGTGCATGCAGCCTAAC
AACATCATGGGCATCGAGAACATTTGCGAACTGGCCGCACGGATGCTC
TTCAGCGCCGTTGAGTGGGCCCGGAACATCCCCTTCTTCCCTGACCTG
CAGATCACGGACCAGGTGGCCCTCCTTCGCCTCACCTGGAGCGAGCTG
TTCGTGTTGAATGCGGCCCAGTGCTCCATGCCCCTCCATGTCGCCCCGC
TCCTTGCCGCTGCTGGCCTGCACGCTTCACCCATGTCAGCCGACCGGG
TGGTCGCTTTTATGGACCACATACGGATCTTCCAAGAGCAAGTGGAGA
AGCTCAAGGCACTGCACGTCGACTCCGCCGAGTATAGCTGCCTCAAGG
CCATAGTCCTGTTCACCTCAGATGCCTGTGGTCTGTCTGATGTAGCCCA
TGTGGAAAGCTTGCAGGAAAAGTCCCAGTGTGCTTTGGAAGAGTACGT
TAGGAGCCAGTACCCCAACCAGCCAACACGGTTCGGAAAGCTCTTGCT
TCGTCTCCCTTCCCTCCGCACGGTCTCCTCCTCAGTCATAGAGCAATTG
TTTTTCGTCCGTTTGGTAGGTAAAACCCCCATCGAAACCCTCATCCGGG
ATATGTTACTGTCCGGCAGCAGTTTTAACTGGCCATATATGGCAATTC
AATAAATAAATCAATCAAAATAAGGGGGAGTGAAACAGAGAAAGAA
AAGGCAAAAGACTGGTTTTGTTTGCTTAATTTCCTTCTGTTAAGAAAG
GATGTTACAAGTTTGCTAAAAAGAAGAGAGGGGAAGAATTTAATGGA
CTGTGAATTTCAAAAAGGAGAGAGAGAAAGAGAGAGACTGCCAAATG
AACTTTTACAGAATGCATTAAAAAAAAAGAAAGAAAACAACTCCTGT
GTTGGGCAGAACAACCTGCTACTTATCATTTTTGTATAAAAAGGAAAT
TAGTCTTTTTTTCTTTTTGGTAAATTTTTGAAAAATATTGCTAAAAGTG
CATTTAAGGAGATTGGGAGAAAATTAGCAGAATGGACAAAGTAAGTC
ATTTTTTTCCAAATTATTAATTGTCCTGTGTCTATGTACCTCTAGTTGTT
CTTTTTTTTTTTTTTTAACTTTTCTGGTTCCAAACCAGTTTATTCTGTGG
TTCAATAATAAGTTTTGATATAATCTTGGCTTCTTAAAAACTGTGTATC
ATTAAAATATATGTTCTGCAAGAATTAAAACTGAGTCCATGAAAATAG
CATAGGAAAACATAAAACTTTAAAAGGCAACTCAGAGATGGTGGAAA
TGCACTTACAAGTGGTGGCCAAATTGTTTTTTTTTTTTTTTTTTTAAGGT
AAAGTTGAGCACTCTAATTAGCAAGCTGGGGGAATCACATCAACACTT
AGCTTCCCCACCCCCACCCCATACCATGACAAACCTAGCTTTTTAAAA
AAAATATTTTAAGAAACAGAAGGAACTGTGGAATTTATTGGCAGCCA
AGGAATGTGTCCAAGACACAAGCTGAGGTTTTTGAATAAAAAGTGAA
CTTTTGTAATTTGAATTGGGTCCCCCCCCCTTAGTTCTTGAATTGTTAT
GAATCCTATATCTGTTTGTATATTTGCAAGCCCTTTGTATTATAATTGT
TGATATTTCCCCTTTTTAAAAAATACCATTGAAATCAGCATGACAAAA
TAACACTGTTGGCACTTATAGGTAACGTGATTGATTCAGTATCTTAGA
GTTTACAGTTTGTGTTTTTAAAAAACTGAAGGTTTTTTTTTTAAGTGCA
ACATTTCTGTATACTGTAAAAGTTATAATAACTGAACTGTTTGGTCGA
GTCTTTGTGTGTTATATTCCAAGGAAATTGAAAGTATTCAGAAATTAA
AATATTATTTGATATCTGAAATCTGCTTGGCTGTCCCCACTCACTGTCT
TTCCACGGAGCTGAGCCCCTGTGAGTTCTCGCTGAGCCAGCGGGGGCC
CCATTTGTTTACTCCCTCAATCAGTTTGTTCAAAGGTAGACTAGTGTAT
TTGCCTGTTTAATTTGGGTGTGGTGTGGGGGGGGAGCTGAAGTTAATG
GTTTAGCTATGGTTTAGGAAGTGCCACACTGATATAGTAAGCCACCCC
CATTCACCTAATCCTACTTTTAATTAAAAATGGATTTTCCAGGAAAAA
AATAAGGCCCTTATATTTGTCACACTTAAGTGCCTGCTTAGGGAAGGT
ATTGTGAAAAGTATTAGAAATTTTGAGATCAGTATCTGTTTTATGATCA
GAAAAAAAATGCTCTTTTGTACATTTGTGACAGTTATGCAGAGGACTG
TCCAAGCAAGCTAATCACAGAACTGTAAATAGAGGGCAGTTGTTTGCA
ATGAGTTTTTCCTTAAGTAAGTGTAATTTTTCTTTTTCTTTTTTTCTTTT
TTTTTTAAAAATATCCTGAGGTTCTCATTTAGCGTGGCTGTTGAGAGGAT
TTTGAATACAGTGATGTAGCTGCTAGCGACGAAGGGTCTGTTTTTCTTG
TATATACATGATAACTTGCAGTTGCCCTGCCTTTCCCCTCCCCCTCCCT
CTTCAGTCTGTTGAGAGCATGGCCACAGGTCAAGGGAATCTTTACCAT
TGGAGTTATGTACATAAAAAAAAAAAACCATGAACATTTGGACATTTC
AGATTATATAGAAACAATCTGTACTGCTCTGGGATCCTTTGGTCTTAG
AAACCATTTTTGGGGGGGTGGAGAGAGAGAGAGGGAGAGGAGAGAG
AGAGAGAGAGAGAGAGAGAGAGAGAGAGAGAGAGAGAGAGAGAGA
GAGAGAGAGAATAAAGAAAACTTTACAGGGTGTGCTGATTTGGGAAG
TCAACTATTTGGTTCTGTCCCTTATTCTCTTTTCCTGGTTTGGGAAGAG
CTCACTGCTGGGAACTTGCAATTTGTTCTTATTTAGACTTTCCAAGCTG
CCCTCCCTGACAATACTTTTACCATGTTGTGGTTTAATCTTAAAACGGG
GGAGGGGGCTGGTGACAGAGGTGAAAGAAAGGAGATCAGTTTGCCAA
GTGCATTCAACTTTGATGCTCAGTTCTGGTTCATACATGCAGACCTGAA
AACTCTGCCTGATTTAGGCAGAGATCTTTATCTGACCCTCAGCTTCCCT
CTGTAGATATATAGATATATAAATATAAATATGAATATAAGTATGTTT
TACAGCACAGCATCTGACCTGTAGATGGAGGTTTTGTTGGTTGTTTATT
TTCCCCTCTTGCAAGTGCTACCCATGTGAGTGTGTGAAGTTTCTCTACT
AAGTAAAACACAGGCCCTTTTCCTTGTTTGCTTTGTGTTAGCTTATTGT
AAACAGCCATTTGTTGTAAATTATTATTGGCATTAAATTATAATTTATG
ATTTTCAAAGC
SEQ ID NO: 8
PREDICTED: Mus musculus nuclear receptor
subfamily 2, group F, member 2 (Nr2f2),
transcript variant X2, mRNA
NCBI Reference Sequence: XM_006540578.1
>gi|568947226|ref|XM_006540578.1| PREDICTED:
Mus musculus nuclear receptor subfamily 2,
group F, member 2 (Nr2f2), transcript
variant X2, mRNA
AAAAAGTGCCTCAAAGTGGGCATGAGACGGGAAGGTATCGGCCTCTC
ATTTCTTCCCCCTTCGCCCGCGGTCCCGGGGCTCTGGGTGCGTTTGGCT
AGCCTGCTCTGGCTGTACAGAGAGGCAGGATGCCTCCTACCCAGCCTA
CCCACGGGCAGTTTGCCCTGACCAACGGGGACCCCCTCAACTGCCACT
CGTACCTGTCCGGATATATTTCCCTGCTGCTGCGCGCGGAGCCCTACC
CCACGTCGCGCTTCGGCAGTCAGTGCATGCAGCCTAACAACATCATGG
GCATCGAGAACATTTGCGAACTGGCCGCACGGATGCTCTTCAGCGCCG
TTGAGTGGGCCCGGAACATCCCCTTCTTCCCTGACCTGCAGATCACGG
ACCAGGTGGCCCTCCTTCGCCTCACCTGGAGCGAGCTGTTCGTGTTGA
ATGCGGCCCAGTGCTCCATGCCCCTCCATGTCGCCCCGCTCCTTGCCGC
TGCTGGCCTGCACGCTTCACCCATGTCAGCCGACCGGGTGGTCGCTTT
TATGGACCACATACGGATCTTCCAAGAGCAAGTGGAGAAGCTCAAGG
CACTGCACGTCGACTCCGCCGAGTATAGCTGCCTCAAGGCCATAGTCC
TGTTCACCTCAGATGCCTGTGGTCTGTCTGATGTAGCCCATGTGGAAA
GCTTGCAGGAAAAGTCCCAGTGTGCTTTGGAAGAGTACGTTAGGAGCC
AGTACCCCAACCAGCCAACACGGTTCGGAAAGCTCTTGCTTCGTCTCC
CTTCCCTCCGCACGGTCTCCTCCTCAGTCATAGAGCAATTGTTTTTCGT
CCGTTTGGTAGGTAAAACCCCCATCGAAACCCTCATCCGGGATATGTT
ACTGTCCGGCAGCAGTTTTAACTGGCCATATATGGCAATTCAATAAAT
AAATCAATCAAAATAAGGGGGAGTGAAACAGAGAAAGAAAAGGCAA
AAGACTGGTTTTGTTTGCTTAATTTCCTTCTGTTAAGAAAGGATGTTAC
AAGTTTGCTAAAAAGAAGAGAGGGGAAGAATTTAATGGACTGTGAAT
TTCAAAAAGGAGAGAGAGAAAGAGAGAGACTGCCAAATGAACTTTTA
CAGAATGCATTAAAAAAAAAGAAAGAAAACAACTCCTGTGTTGGGCA
GAACAACCTGCTACTTATCATTTTTGTATAAAAAGGAAATTAGTCTTTT
TTTCTTTTTGGTAAATTTTTGAAAAATATTGCTAAAAGTGCATTTAAGG
AGATTGGGAGAAAATTAGCAGAATGGACAAAGTAAGTCATTTTTTTCC
AAATTATTAATTGTCCTGTGTCTATGTACCTCTAGTTGTTCTTTTTTTTT
TTTTTTAACTTTTCTGGTTCCAAACCAGTTTATTCTGTGGTTCAATAATA
AGTTTTGATATAATCTTGGCTTCTTAAAAACTGTGTATCATTAAAATAT
ATGTTCTGCAAGAATTAAAACTGAGTCCATGAAAATAGCATAGGAAA
ACATAAAACTTTAAAAGGCAACTCAGAGATGGTGGAAATGCACTTAC
AAGTGGTGGCCAAATTGTTTTTTTTTTTTTTTTTTTAAGGTAAAGTTGA
GCACTCTAATTAGCAAGCTGGGGGAATCACATCAACACTTAGCTTCCC
CACCCCCACCCCATACCATGACAAACCTAGCTTTTTAAAAAAAATATT
TTAAGAAACAGAAGGAACTGTGGAATTTATTGGCAGCCAAGGAATGT
GTCCAAGACACAAGCTGAGGTTTTTGAATAAAAAGTGAACTTTTGTAA
TTTGAATTGGGTCCCCCCCCCTTAGTTCTTGAATTGTTATGAATCCTAT
ATCTGTTTGTATATTTGCAAGCCCTTTGTATTATAATTGTTGATATTTCC
CCTTTTTAAAAAATACCATTGAAATCAGCATGACAAAATAACACTGTT
GGCACTTATAGGTAACGTGATTGATTCAGTATCTTAGAGTTTACAGTTT
GTGTTTTTAAAAAACTGAAGGTTTTTTTTTTAAGTGCAACATTTCTGTA
TACTGTAAAAGTTATAATAACTGAACTGTTTGGTCGAGTCTTTGTGTGT
TATATTCCAAGGAAATTGAAAGTATTCAGAAATTAAAATATTATTTGA
TATCTGAAATCTGCTTGGCTGTCCCCACTCACTGTCTTTCCACGGAGCT
GAGCCCCTGTGAGTTCTCGCTGAGCCAGCGGGGGCCCCATTTGTTTAC
TCCCTCAATCAGTTTGTTCAAAGGTAGACTAGTGTATTTGCCTGTTTAA
TTTGGGTGTGGTGTGGGGGGGGAGCTGAAGTTAATGGTTTAGCTATGG
TTTAGGAAGTGCCACACTGATATAGTAAGCCACCCCCATTCACCTAAT
CCTACTTTTAATTAAAAATGGATTTTCCAGGAAAAAAATAAGGCCCTT
ATATTTGTCACACTTAAGTGCCTGCTTAGGGAAGGTATTGTGAAAAGT
ATTAGAAATTTTGAGATCAGTATCTGTTTTATGATCAGAAAAAAAATG
CTCTTTTGTACATTTGTGACAGTTATGCAGAGGACTGTCCAAGCAAGC
TAATCACAGAACTGTAAATAGAGGGCAGTTGTTTGCAATGAGTTTTTC
CTTAAGTAAGTGTAATTTTTCTTTTTCTTTTTTTCTTTTTTTTTTAAAAA
TATCCTGAGGTTCTCATTTAGCGTGGCTGTTGAGAGGATTTTGAATACA
GTGATGTAGCTGCTAGCGACGAAGGGTCTGTTTTTCTTGTATATACATG
ATAACTTGCAGTTGCCCTGCCTTTCCCCTCCCCCTCCCTCTTCAGTCTG
TTGAGAGCATGGCCACAGGTCAAGGGAATCTTTACCATTGGAGTTATG
TACATAAAAAAAAAAAACCATGAACATTTGGACATTTCAGATTATATA
GAAACAATCTGTACTGCTCTGGGATCCTTTGGTCTTAGAAACCATTTTT
GGGGGGGTGGAGAGAGAGAGAGGGAGAGGAGAGAGAGAGAGAGAG
AGAGAGAGAGAGAGAGAGAGAGAGAGAGAGAGAGAGAGAGAGAGA
ATAAAGAAAACTTTACAGGGTGTGCTGATTTGGGAAGTCAACTATTTG
GTTCTGTCCCTTATTCTCTTTTCCTGGTTTGGGAAGAGCTCACTGCTGG
GAACTTGCAATTTGTTCTTATTTAGACTTTCCAAGCTGCCCTCCCTGAC
AATACTTTTACCATGTTGTGGTTTAATCTTAAAACGGGGGAGGGGGCT
GGTGACAGAGGTGAAAGAAAGGAGATCAGTTTGCCAAGTGCATTCAA
CTTTGATGCTCAGTTCTGGTTCATACATGCAGACCTGAAAACTCTGCCT
GATTTAGGCAGAGATCTTTATCTGACCCTCAGCTTCCCTCTGTAGATAT
ATAGATATATAAATATAAATATGAATATAAGTATGTTTTACAGCACAG
CATCTGACCTGTAGATGGAGGTTTTGTTGGTTGTTTATTTTCCCCTCTT
GCAAGTGCTACCCATGTGAGTGTGTGAAGTTTCTCTACTAAGTAAAAC
ACAGGCCCTTTTCCTTGTTTGCTTTGTGTTAGCTTATTGTAAACAGCCA
TTTGTTGTAAATTATTATTGGCATTAAATTATAATTTATGATTTTCAAA
GCAAAA
SEQ ID NO: 9
Protein Sequence of human NR2F2
Homo sapiens nuclear receptor subfamily 2,
group F, member 2 (NR2F2), protein from
transcript variant 1
MAMVVSTWRDPQDEVPGSQGSQASQAPPVPGPPPGAPHTPQTPG
QGGPASTPAQTAAGGQGGPGGPGSDKQQQQQHIECVVCGDKSSGKHYG
QFTCEGCKSFFKRSVRRNLSYTCRANRNCPIDQHHRNQCQYCRLKKCLK
VGMRREAVQRGRMPPTQPTHGQFALTNGDPLNCHSYLSGYISLLLRAEP
YPTSRFGSQCMQPNNIMGIENICELAARMLFSAVEWARNIPFFPDLQITD
QVALLRLTWSELFVLNAAQCSMPLHVAPLLAAAGLHASPMSADRVVAFM
DHIRIFQEQVEKLKALHVDSAEYSCLKAIVLFTSDACGLSDVAHVESLQE
KSQCALEEYVRSQYPNQPTRFGKLLLRLPSLRTVSSSVIEQLFFVRLVGK
TPIETLIRDMLLSGSSFNWPYMAIQ
SEQ ID NO: 10
Protein Sequence of human NR2F2
Homo sapiens nuclear receptor subfamily 2,
group F, member 2 (NR2F2), protein from
transcript variant 2
MQAVWDLEQGKYGFAVQRGRMPPTQPTHGQFALTNGDPLNCHSYLSGY
ISLLLRAEPYPTSRFGSQCMQPNNIMGIENICELAARMLFSAVEWARNIP
FFPDLQITDQVALLRLTWSELFVLNAAQCSMPLHVAPLLAAAGLHASPMS
ADRVVAFMDHIRIFQEQVEKLKALHVDSAEYSCLKAIVLFTSDACGLSDV
AHVESLQEKSQCALEEYVRSQYPNQPTRFGKLLLRLPSLRTVSSSVIEQL
FFVRLVGKTPIETLIRDMLLSGSSFNWPYMAIQ
SEQ ID NO: 11
Protein Sequence of human NR2F2
Homo sapiens nuclear receptor subfamily 2,
group F, member 2 (NR2F2), protein from
transcript variant 3
MPPTQPTHGQFALTNGDPLNCHSYLSGYISLLLRAEPYPTSRFGSQCMQP
NNIMGIENICELAARMLFSAVEWARNIPFFPDLQITDQVALLRLTWSELF
VLNAAQCSMPLHVAPLLAAAGLHASPMSADRVVAFMDHIRIFQEQVEKLK
ALHVDSAEYSCLKAIVLFTSDACGLSDVAHVESLQEKSQCALEEYVRSQY
PNQPTRFGKLLLRLPSLRTVSSSVIEQLFFVRLVGKTPIETLIRDMLLSG
SSFNWPYMAIQ
SEQ ID NO: 12
Protein Sequence of human NR2F2
Homo sapiens nuclear receptor subfamily 2,
group F, member 2 (NR2F2), protein from
transcript variant 4
MPPTQPTHGQFALTNGDPLNCHSYLSGYISLLLRAEPYPTSRFGSQCMQP
NNIMGIENICELAARMLFSAVEWARNIPFFPDLQITDQVALLRLTWSELF
VLNAAQCSMPLHVAPLLAAAGLHASPMSADRVVAFMDHIRIFQEQVEKLK
ALHVDSAEYSCLKAIVLFTSDACGLSDVAHVESLQEKSQCALEEYVRSQY
PNQPTRFGKLLLRLPSLRTVSSSVIEQLFFVRLVGKTPIETLIRDMLLSG
SSFNWPYMAIQ
SEQ ID NO: 13
Protein Sequence of NR2F2 mus musculus
Mus musculus nuclear receptor subfamily 2,
group F, member 2(Nr2f2), protein from
transcript variant 1
MAMVVSTWRDPQDEVPGSQGSQASQAPPVPGPPPGAPHTPQTPGQGGPA
STPAQTAAGGQGGPGGPGSDKQQQQQHIECVVCGDKSSGKHYGQFTCEG
CKSFFKRSVRRNLSYTCRANRNCPIDQHHRNQCQYCRLKKCLKVGMRRE
AVQRGRMPPTQPTHGQFALTNGDPLNCHSYLSGYISLLLRAEPYPTSRFG
SQCMQPNNIMGIENICELAARMLFSAVEWARNIPFFPDLQITDQVALLRL
TWSELFVLNAAQCSMPLHVAPLLAAAGLHASPMSADRVVAFMDHIRIFQE
QVEKLKALHVDSAEYSCLKAIVLFTSDACGLSDVAHVESLQEKSQCALEE
YVRSQYPNQPTRFGKLLLRLPSLRTVSSSVIEQLFFVRLVGKTPIETLIR
DMLLSGSSFNWPYMAIQ
SEQ ID NO: 14
Protein Sequence of NR2F2 mus musculus
Mus musculus nuclear receptor subfamily 2,
group F, member 2(Nr2f2), protein from
transcript variant 1X
MGLTAVQRGRMPPTQPTHGQFALTNGDPLNCHSYLSGYISLLLRAEPYPT
SRFGSQCMQPNNIMGIENICELAARMLFSAVEWARNIPFFPDLQITDQVA
LLRLTWSELFVLNAAQCSMPLHVAPLLAAAGLHASPMSADRVVAFMDHIR
IFQEQVEKLKALHVDSAEYSCLKAIVLFTSDACGLSDVAHVESLQEKSQC
ALEEYVRSQYPNQPTRFGKLLLRLPSLRTVSSSVIEQLFFVRLVGKTPIE
TLIRDMLLSGSSFNWPYMAIQ
SEQ ID NO: 15
Protein Sequence of NR2F2 mus musculus
Mus musculus nuclear receptor subfamily 2,
group F, member 2(Nr2f2), protein from
transcript variant 2
MQAVWDLEQGKYGFAVQRGRMPPTQPTHGQFALTNGDPLNCHSYLSGY
ISLLLRAEPYPTSRFGSQCMQPNNIMGIENICELAARMLFSAVEWARNIP
FFPDLQITDQVALLRLTWSELFVLNAAQCSMPLHVAPLLAAAGLHASPMS
ADRVVAFMDHIRIFQEQVEKLKALHVDSAEYSCLKAIVLFTSDACGLSDV
AHVESLQEKSQCALEEYVRSQYPNQPTRFGKLLLRLPSLRTVSSSVIEQL
FFVRLVGKTPIETLIRDMLLSGSSFNWPYMAIQ
SEQ ID NO: 16
Protein Sequence of NR2F2 mus musculus
Mus musculus nuclear receptor subfamily 2,
group F, member 2(Nr2f2), protein from
transcript variant 2X
MPPTQPTHGQFALTNGDPLNCHSYLSGYISLLLRAEPYPTSRFGSQCMQP
NNIMGIENICELAARMLFSAVEWARNIPFFPDLQITDQVALLRLTWSELF
VLNAAQCSMPLHVAPLLAAAGLHASPMSADRVVAFMDHIRIFQEQVEKLK
ALHVDSAEYSCLKAIVLFTSDACGLSDVAHVESLQEKSQCALEEYVRSQY
PNQPTRFGKLLLRLPSLRTVSSSVIEQLFFVRLVGKTPIETLIRDMLLSG
SSFNWPYMAIQ
SEQ ID NO: 17
Human NR2F2 siRNA 1 GCCGUCUCAAGAAGUGCUU
SEQ ID NO: 18
Human NR2F2 siRNA 2 CAUUGAGACACUGAUCAGA
SEQ ID NO: 19
Human NR2F2 siRNA 3 GCAAGCAUUACGGUGUCUU
SEQ ID NO: 20
Human NR2F2 siRNA 4 CCCCUAGCAUGAACUUGUG
Primers
Human NR2F2 pair1:
SEQ ID NO: 21
Fwd: TGGTCGCCTTTATGGACCAC
SEQ ID NO: 22
Revs: GCGAAGCAAAAGCTTTCCGA
Human NR2F2 pair2:
SEQ ID NO: 23
Fwd: 5′-GGAGCGAGCTGTTTGTGTTG-3′
SEQ ID NO: 24
Revs: 5′-TGGTCCATAAAGGCGACCAC-3′
Human NR2F2 pair3:
SEQ ID NO: 25
Fwd: 5′-TCGGAAAGCTTTTGCTTCGC-3′
SEQ ID NO: 26
Revs: 5′-GGCCAGTTAAAACTGCTGCC-3′
Human GAPDH:
SEQ ID NO: 27
Fwd: 5′-GGCCTCCAAGGAGTAAGACC-3′
SEQ ID NO: 28
Revs: 5′-AGGGGTCTACATGGCAACTG-3′
3′ end Mus NR2F2 pair 1:
SEQ ID NO: 29
Fwd: 5′-AAACCCCCATCGAAACCCTC-3′
SEQ ID NO: 30
Revs: 5′-AGTAGCAGGTTGTTCTGCCC-3′
3′ end Mus NR2F2 pair 2:
SEQ ID NO: 31
Fwd: 5′-CAGGGTGTGCTGATTTGGGA-3′
SEQ ID NO: 32
Revs: 5′-GTTCCCAGCAGTGAGCTCTT-3′
3′ end Mus NR2F2 pair 3:
SEQ ID NO: 33
Fwd: 5′-GCAGAGGACTGTCCAAGCAA-3′
SEQ ID NO: 34
Revs: 5′-CCTCTCAACAGCCACGCTAA-3′
3′ end Mus L32:
SEQ ID NO: 35
Fwd: 5′-GCCATCAGAGTCACCAATCC-3′
SEQ ID NO: 36
Revs: 5′-AAACATGCACACAAGCCATC-3′

1. A method of treating a myelodysplastic syndrome comprising the steps of: a) identifying a patient suffering from a myelodysplastic syndrome; b) administering to said patient an inhibitor of NR2F2 gene; c) assessing reduction in disease burden; and d) adjusting dose of said NR2F2 inhibitor based on response achieved compared to desired response.

2. The method of claim 1, wherein said inhibitor of NR2F2 is a mRNA sequence of at least 75% sequence identity to the mRNA sequence of SEQ ID NO: 1, 2, 3 or 4 that induces the RNA interference, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:1, 2, 3 or 4 that has been selected based on its ability to suppress the expression of NR2F2 by induction of RNA interference.

3. The method of claim 2, wherein said inhibitor is selected from a group comprising of: a) a chemically synthesized double stranded siRNA; b) a short-hairpin ribonucleic acid (shRNA) molecule; c) an antisense ribonucleic acid molecule

4. The method of claim 1, wherein said assessment of desired response is achieved through quantification of mature blood cells in circulation of said patient.

5. The method of claim 1, wherein said assessment of desired response is achieved through histological examination of said patient bone marrow for changes associated with reversion of MDS.

6. A method of claim 1, wherein said inhibitor inhibits expression of NR2F2 protein in a patient suffering from MDS for a therapeutic purpose of substantially inhibiting MDS, and/or reducing the rate of transformation of MDS to a leukemic state

7. The method of claim 1, wherein the pharmaceutical composition further comprises a delivery agent.

8. The method of claim 1, wherein the delivery agent comprises a liposome.

9. A method of claim 1, wherein said inhibitor inhibiting growth of cells giving rise to MDS.

10. The method of claim 1, wherein the step of contacting the cells that give rise to MDS with the siRNA results in at least one of an induction of differentiation or decreased MDS stem cell activity indicated by a decrease in one of the following self-renewal, growth, proliferation, differentiation and programmed cell death in mammalian cells.

11. The method of claim 1 wherein the effective portion of the oligonucleotide consists of SEQ ID NO: 17-20.

12. A composition useful for treatment of MDS or inhibiting progression to leukemia in an MDS patient, or a preleukemic patient comprising an oligonucleotide complementary to a nuclear receptor having a mRNA sequence of at least 75% sequence identity to the mRNA sequence of SEQ ID NO: 1, 2, 3 or 4 wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:1, 2, 3 or 4 that is selected based on its ability to inhibits the expression of the nuclear receptor NR2F2 by causing degradation of a ribonucleic acid encoding nuclear receptor NR2F2.

13. A composition of claim 12 consisting of a short-interfering ribonucleic acid (siRNA) molecule.

14. A composition of claim 12 consisting of a short-hairpin ribonucleic acid (shRNA) molecule.

15. A composition of claim 12 consisting of an antisense ribonucleic acid molecule.

16. A pharmaceutical composition comprising the oligonucleotide of claim 13.

17. The pharmaceutical composition of claim 13 further comprising at least one additional chemotherapeutic agent.

18. The pharmaceutical composition of claim 13 further comprising a delivery agent.

19. The pharmaceutical composition of claim 18, wherein the delivery agent comprises a liposome.

20. A method of growing cells from patients with myelodysplastic syndrome in vitro, consisting of placing cells from a patient with myelodysplastic syndrome in a culture containing an OP9 feeder layer and liquid medium containing at least three of the following growth factors: TPO, IL-11, Flt-3 ligand, G-CSF, GM-CSF, SCF, insulin, transferring, BSA Fraction V, and FBS.
